(19) United States
(12) Patent Application Publication
(10) Pub. No.: US 2013/0197296 A1
(43) Pub. Date: Aug. 1, 2013

(54) Removing Cells from an Organism

(71) Applicants: Karl-Heinz Ott, Lawrenceville, NJ (US); Johannes Dapprich, Lawrenceville, NJ (US)

(72) Inventors: Karl-Heinz Ott, Lawrenceville, NJ (US); Johannes Dapprich, Lawrenceville, NJ (US)

(21) Appl. No.: 13/740,817

(22) Filed: Jan. 14, 2013

Related U.S. Application Data
(60) Provisional application No. 61/586,461, filed on Jan. 13, 2012.

(51) Int. Cl. A61N 2/00; A61M 3/00; A61N 2/06

(52) U.S. Cl. CPC A61N 2/004; A61N 2/06; A61M 3/00
USPC 600/12

(57) ABSTRACT

A minimally invasive method to eliminate circulating agents from an organism to prevent and treat diseases is disclosed. The method utilizes immunomagnetic methods to concentrate and localize disease-associated agents in a small region of the body. Subsequently, the complexes are removed from the body. Removal of disease-causing or disease-promoting agents (circulating tumor cells, bacteria, viruses and virus-infected cells, certain immune cells) would add a significant new option for intervention of disease progression and supplement other therapeutic options.

### CROSS-REFERENCE TO RELATED APPLICATIONS

This application claims priority to U.S. Provisional Application No. 61/586,461, filed Jan. 13, 2012, which is herein incorporated by reference in its entirety.

### BACKGROUND

An organism, in particular, body fluids and body cavities of humans and animals may carry a wide variety of disease-causing, disease-modifying, or otherwise unwanted agents (herein called disease-associated circulating entities, DACEs). Elimination of such agents can help treat diseases associated with such agents.

This invention relates to managing specific diseases, particularly metastatic cancers. The present invention is the specified use of nanostructures. The present invention is a medical treatment for cancer, immunological and infectious diseases.

Disease associated agents, for example disease associated cells, are generally rare in a given body fluid and comprise a small percentage of the total number of endogenous molecules or cells. Therefore, most therapeutic techniques focus on systemic treatments, including the use of drugs to eliminate, inactivate or destroy these cells in-vivo. In the case of cancer cells, for example, chemotherapy or immunotherapies are common techniques. In the case of bacteremia, antibiotics may be used against circulating bacteria. Furthermore, it is sometimes desirable to isolate large quantities of specific agents from an organism, e.g., for research or diagnostic purposes.

These approaches, however, are not completely effective due in part to increasingly resistant strains of bacteria or the escape of cancer cells from systemic treatments. Systemic treatments are often invasive and may cause severe side effects. Local treatments, such as surgical removal of a tumor, can in most cases not be used to eliminate DACE-associated diseases. Despite efforts of early diagnosis and prevention¹, lung, colorectal, stomach, liver and breast cancers are associated with the highest number of mortalities². Most of the morbidity and mortality from cancer is caused by metastases in distant sites such as lymph nodes, bone, lung, liver, and brain^{3, 4}.

Systemic and local therapies, while providing many benefits to patients, have made little progress in curing metastatic disease.^{5,6,7,8,9}. In developed countries, nearly 30% of women with early stage breast cancer will eventually develop metastatic breast cancer. Metastasis reduces the rate of survival from ˜92% for women with localized tumors that can be removed down to 73% for woman who have circulating tumor cells (CTCs) in their lymph nodes at the time of diagnosis, and down to 13% if metastases have already established themselves

Metastases form when CTCs from a primary tumor enter distal tissues and form secondary, aggressive tumors¹⁰. CTCs can both seed new and maintain existing tumors^{11, 12, 13}. CTCs can be identified by the presence of, amongst others, epithelial cell adhesion molecule (EpCAM)^{14, 15, 16}. The metastatic process begins when cancer cells enter the circulatory system through newly formed, imperfect and leaky blood vessels of the tumor. CTCs travel through the circulatory system and, if they escape the immune system, can invade tissues to form micro-metastases that can develop into secondary, metastatic tumors.

A large number of clinical studies has demonstrated^{(17,18,19,20,21,22,23,24,25,26)} that:

1) poor outcome after surgery and chemotherapy is observed when CTCs remain in the body,
2) the number of CTCs in the blood is highly correlated with disease progression,
3) blood vessels and the lymphatic system act as conduits to transport malignant circulating tumor cells¹³.

Clinical and preclinical evidence^{(17,18,19,20,21,22,23,24,25,26)} indicates that a reduction of DACE count through the depletion or removal of DACEs from circulation can be a method for treating diseases that otherwise lack effective and safe treatment. As such, removing DACEs from circulation could overcome a number of diseases without the need to identify specific and potentially harmful drugs. This would not only be a major improvement in management of diseases, but could also have a major impact on global public health by increasing availability and reducing cost.

In addition to the benefit to the individual patient, simple and broadly applicable methods to reduce the incidence of diseases caused by DACEs would generate economic benefits by lowering health care costs and thus enabling access to treatment for more patients worldwide. For example, economic, regional, and race disparities significantly impact cancer rates²⁷. Access to inexpensive and simple treatment options could therefore make a significant impact for cancer patients worldwide.

Immunomagnetic techniques for the enrichment and detection DACEs have been practiced for decades, e.g. U.S. Pat. No. 3,970,518 describes “small magnetic particles coated with an antibody layer are used to provide large and widely-distributed surface area for sorting out and separating select viruses, bacteria and other cells from multi-cell, bacteria or virus populations”. Following, numerous nanoparticle designs have been disclosed that can be utilized to selectively bind and complex with cells or other DACEs. Such particles are commercially available.

Immunomagnetic separation has become a subject of much research, and diagnostic approaches have generated substantial knowledge about the specific surface structures (epitopes) that characterize certain pathogens and complementary antibodies and similar molecules.

Similar methods using nanodevices coated with EpCAM antibodies or the ex-vivo isolation of CTCs through immunomagnetic particles and small magnets applied to very small reaction volumes have been optimized to isolate small numbers of CTCs with the majority of CTCs remaining largely undisturbed and therefore being available for molecular profiling analysis. However, these methods typically require extensive equilibration times, cannot process all the blood volume of a patient and are thus only useful for ex-vivo diagnostic, not therapy.

As such, methods to create particles and devices to specifically bind and identify DACEs focus on the enrichment and/or extraction of these cells for the purpose of diagnostic identification or characterization of a disease. While many of these devices are thought to help in the treatment of cancers, these methods currently all rely on the paradigm that CTC-based treatments will consist of isolating CTCs followed by molecular profiling as a diagnostic tool to choose the most suitable treatment option. However two major roadblocks exist: 1) such profiling methodology has yet to be validated to yield suitable molecular targets. 2) Even if targets were identified, the process would still require the successful discovery, development and clinical testing of potentially suitable drugs or drug combinations—which is an arduous, time-consuming, expensive and uncertain task. As such, methods of counting or isolating DACEs ex-vivo do not accomplish the healing of a patient.

The quantification of CTCs can for instance be accomplished with an automated cell enrichment and immunocytochemical detection system such as the CellSearch System by Veridex, Warren, N.J. In this system, circulating epithelial cells are isolated from a small sample (7.5 ml) of blood by antibody-coated magnetic particles in a magnetic field and identified using a semi-automated fluorescence microscope.

The in vivo use of nanoparticles, and in particular paramagnetic and superparamagnetic nanoparticles, has been studied in the context of in vivo imaging where nanoparticles are carriers of contrast agents (e.g. carriers for PET contrast agents) or of drugs, i.e. drug delivery. The iron content of such particles can be visualized via magnetic resonance imaging. For example, Feridex I.V.® (ferumoxides injectable solution) is a sterile aqueous colloid of superparamagnetic iron oxide associated with dextran for intravenous (i.v.) administration as a magnetic resonance imaging contrast media. The utility of such particles for a variety of therapeutic and diagnostic uses has widely been discussed²⁸⁻³⁰.

U.S. Patent application 2007/0275007 discloses a number of nanoparticle functionalizations and methods to generate biocompatible nanoparticles, and is hereby incorporated in its entirety. U.S. patent application Ser. No. 13/109,425 describes aspects of making nanoparticles designed to bind to cancer cells, and is hereby incorporated as reference in its entirety.

U.S. Patent Application 2012/0004293 describes aspects of making nanoparticles designed to bind to cancer cells. U.S. Pat. No. 6,656,587 describes coated nanoparticles with reduced self-adhesion.

Given the long-standing interest, there is also a large body of literature describing methods for the production of suitable nanoparticles, parameters influencing their biological compatibility, suitable coatings and functionalization³⁸⁻⁵¹. Similarly, evaluations of nanoparticles and their interaction with biological systems can be accomplished by, e.g. fluorescence microscopy, and fluorescent-based cell sorting techniques (FACS) in vitro, and pharmacokinetics and histology evaluations in vivo, which are all well-established methods that have been used for the evaluation of magnetic nanoparticles for e.g. cell uptake of nanoparticles and in vivo ditribution⁵¹⁻⁵⁵.

The use of exogenous nanoparticles that target specific ligands on a solid tumor may allow an increased level of selectivity of ablation by directing the particles to specific types of cells or a specific location within the body. U.S. Pat. Nos. 6,344,272 and 6,685,986 teach the compositions and synthesis of one class of nanoparticles. U.S. Pat. Nos. 5,385,707 and 6,417,011 teaches methods to produce immunomagnetic nanoparticles directed against specific targets. U.S. Pat. No. 6,530,944 describes localized in-vivo treatments of cells or tissues by the delivery of nanoparticles to said cells or tissues. This treatment is applicable to a stationary solid tumor mass. U.S. Pat. No. 7,285,412 describes magnetic capture of cells using a microdevice.

High gradient magnetic separation (HGMS) of biologicals is reviewed and discussed in several U.S. patents including U.S. Pat. No. 5,385,707 (Miltenyi et al., 1995), U.S. Pat. No. 5,541,072 (Wang et al., 1996) and U.S. Pat. No. 5,646,001 (Terstappen et al., 1997), U.S. Pat. No. 6,365,362 (Terstappen et al., 2002), all of which are incorporated herein by reference. U.S. Pat. No. 5,385,707 discloses a process for preparing superparamagnetic colloidal coated particles for use in HGMS. The process comprises precipitating magnetic iron oxide (from ferric/ferrous ion solution) in colloidal form, treating the colloid with a suitable coating material such as dextran, and thereafter derivatizing (conjugating) the coated magnetic particles to a specificity-conferring moiety such as avidin or biotin.

The use of biotin conjugates (such as biotinylated monoclonal antibodies) and avidin/streptavidin conjugates (such as radioactive streptavidin) for tumor detection, imaging and therapy is also disclosed in the prior art. See U.S. Pat. No. 5,482,698 (Griffiths, 1996) and the patents and literature references cited therein.

Improvements to separation by nonspecific aggregation of the coating have been described in U.S. Pat. No. 6,620,627, bioactivation is described in U.S. Pat. No. 7,998,923, marking particle with fluorescent dyes is disclosed in U.S. Pat. No. 7,198,847.

Other nanoparticles have been described for the in vivo ablation of solid tumors and tissues. For example, paramagnetic particles, gold nanorods and carbon nanotubes have been described—generally with targeting ligands—for the ablation of solid tumors and tissue. These particles have been delivered intravenously or by direct injection into the tumor. These techniques are applicable to solid tumors, but they have generally not been useful for the ablation or elimination of cells circulating in blood or the lymphatic system, or of cells residing in various body cavities such as the bone marrow.

Nanoparticles specifically for in vivo use have also been described⁵⁶ and utilized for ex vivo removal of DACE from ascites fluid^{57,58}.

Extracorporeal devices have also been incorporated in other biological processes and methods. For example, dialysis, or the membrane-based separation of blood components, is a common medical procedure. These techniques are not designed for the treatment of specific cells in the blood, but rather provide for the removal of proteins and molecules normally removed by properly functioning body organs. Apheresis of proteins has also been described for the treatment of diseases, such as dry macular degeneration. These techniques do not treat circulating cells, much less specifically targeted cells, during the process.

United States Patent Publication No. 2004/0191246 describes an implantable two chamber device for the separation of biological cells. The application describes the use of the separated cells for immunotherapy and other means by the in-vivo treatment of bodily fluid, and also makes reference to the “neutralization” of such cells, but does not describe the methods for such neutralization, nor does it describe how such methods distinguish between the target and the remaining blood cells. Additionally, this application contemplates separating the targets from the remaining blood components within the device.

Various devices have been developed for the separation or enrichment of specific cells from samples of body fluid, yet these devices are limited to operating on only the sample itself and are not capable to treat the entire blood component of a patient. U.S. Patent Publication No. 2006/0252087 describes methods for the separation of cells or target molecules from a body fluid sample. U.S. Patent Publication No. 2006/0141045 describes beads that may be used for cell separation from body fluid samples. Other examples are also described in the literature. These devices, however, are designed and limited to utilize only a small fluid sample and therefore are not useful for treating the entire blood volume of a patient.

Accordingly, improved methods are needed that address one or more disadvantages of the prior art. U.S. Pat. No. 5,104,373 describes a method for extracorporeally treating blood samples by one or all of several modalities, including (i) the hyperthermic treatment of blood at a reduced pH; (ii) mechanically damaging or lysing blood cells that contain or have been affected by a virus, microorganism or disease state, and so as to render them more fragile than other cells; and (iii) subjecting the blood to irradiation. This device, however, is not selective in its application of the various treatments to specific cell types or blood components of the patient. Disadvantages of these techniques therefore include the failure to preferentially treat the undesirable cell subpopulation in the irradiated blood stream, as opposed to treating the entire blood volume.

U.S. Pat. No. 2004/0191246 describes a device for the separation of biological cells. The application describes the use of the separated cells for immunotherapy and other means by the in-vivo treatment of bodily fluid, and also makes reference to the “neutralization” of such cells. However it does not describe methods for such neutralization, nor does it describe how such methods distinguish between the target and the remaining blood cells. Additionally, this application contemplates separating the targets from the remaining blood components within the device. Similar approaches are described in U.S. Pat. Appl. 2010/0167372 and its related applications.

Apheresis and similar techniques are known in the art. For example, U.S. Pat. No. 6,528,057 describes a method for reducing viral load by removal of viruses or fragments or components thereof from the blood by extracorporeally circulating blood through hollow fibers which have in the porous exterior surface, immobilized affinity molecules having specificity for viral components. Passage of the fluid through the hollow fibers causes the viral particles to bind to the affinity molecules so as to reduce the viral load in the effluent.

U.S. Pat. No. 8,057,418 describes devices and methods for extracorporeal ablation of circulating cells by damaging the cells in an extracorporeal device after having injected energy-absorbing nanoparticles to associate preferably with the target cells. Other aspects of extracorporeal Devices and their use are covered in U.S. Patent Applications 2009/0156976.

Herrmann et al.⁵⁹ and U.S. Patent Application 2009/999077 describe the use of extracorporeal devices that magnetically separate tumor cells from the blood or ablate those by transporting the blood through an extracorporeal device wherein magnetic nanoparticles are used to separate target molecules or cells from the blood. Since this approach relies on an extracorporeal device, it requires very rapid binding kinetics and a highly efficient removal of the magnetized targets from the flow within the device. These two requirements contradict one another: Rapid binding kinetics based on diffusional mixing alone requires very small (<<100 nm) nanoparticles. However the need for rapid capture requires relatively large magnetic particles (>500 nm) that are able to generate a sufficiently strong magnetic force to be efficiently moved through a viscous fluid by an externally applied magnetic field. Herrmann et al.⁶⁰ have also described how the direct injection of stable nanomagnets into whole blood ex vivo can be combined with extracorporeal magnetic extraction of chemical compounds for the treatment of severe intoxications, sepsis, metabolic disorders, and autoimmune diseases (removal of pathogenic autoantibodies or immune complexes).

However, any use of extracorporeal devices has to solve the issue of retaining whole blood integrity as a prerequisite for a successful therapeutic application. This requirement severely limits the time during which a sample can make contact with an ablative energy source or a magnetic filter and has turned out to be one of the main reasons for why these devices have as yet failed to become therapeutically useful. Similarly, the requirement to avoid shearing and other mechanical damage to the vital components of a biofluid severely limits certain design specifications of extracorporeal filtration devices, such as surface contact area, surface modification, pumping action, tube length, channel dimensions, Reynolds number, filter pore size and maximum achievable flow rate: Large pores permit the transmission of more sample but fail to capture small nanoparticles. Small particles exhibit a much more limited magnetic field response compared to larger particles (volume and magnetic force drop with the third power of particle size) and therefore are typically not captured from a viscous liquid such as blood under flow conditions. The use of large nanoparticles however limits an effective complex formation because of the large diffusion coefficient of the particles and target cells, which makes mixing inefficient and drives incubation times into hours and days.

Nanoparticles have been tested in vivo as reagents for the imaging of tumors^{61,62,63}. Attempts are also under way to use nanoparticles in vivo to label tumors to target them for destruction by heat or light⁶⁴. Such therapeutic approaches, if effective, may supplement current choices in cancer treatments by attacking larger tumors or metastases but are not expected to be applicable to individually circulating DACEs as they lack the necessary ability to distinguish between DACE and natural cells. However, there were no reports that such particles carried any significant side effects, indicating that properly functionalized nanoparticles (fNPs) may safely be utilized in vivo.

A further medical application of nanoparticles embodies the targeted delivery of drug molecules to a target tissue or cell. In some embodiments magnetic nanoparticles have been employed to direct the therapeutic agent (e.g. a drug) to a certain location such as a tumor.

Hence, it is known that DACEs can be marked by specific antibodies, and antibody-coated nanoparticles can be used ex vivo to isolate or count CTCs in small blood samples. It is also known that fNPs can be used safely in vivo for imaging or drug delivery⁶⁵. Attempts are also under way to use extracorporeal devices to eliminate or destroy circulating tumor cells. However, a minimally invasive method of direct elimination of a wide variety of DACEs has never been conventionalized.

The patents cited in here are all incorporated by reference in their entirety.

### BRIEF SUMMARY OF THE INVENTION

In a first embodiment, the invention is a method for removal of disease-associated circulating entities (DACEs) from an organism. The method comprises the steps of: (1) introducing paramagnetic or superparamagnetic nanoparticles into a fluid component of the organism, wherein the nanoparticles are functionalized so that they comprise at least one bioaffinity molecule that binds to a first target surface structure on a first disease-associated circulating entity and wherein the nanoparticles are introduced into a compartment of the body that permits the nanoparticles to contact the first disease-associated circulating entities, wherein complexes of functionalized nanoparticles (fNPs) and first disease-associated circulating entities form upon contact; (2) applying a magnetic field to a target region of the body, wherein the magnetic field concentrates the nanoparticles within the target region; and (3) removing from the body the complexes within the target region.

In one embodiment of the method, the magnetic field of step 2 is externally applied. In another embodiment, the contacting of the first disease-associated circulating entities and the nanoparticles is enhanced by an additional step of magnetic mixing of the first disease-associated circulating entities and the nanoparticles. In a related embodiment, the magnetic mixing step comprises an externally applied magnetic field wherein the magnetic field causes relative motion of the nanoparticles with respect to the fluid within which the nanoparticles circulate and wherein the intensity of the magnetic field does not immobilize the nanoparticles. In yet another embodiment, the step of applying a magnetic field is accomplished using a magnetic capture device that applies a magnetic field of sufficient intensity within the target region to immobilize the complexes in the fluid.

In another embodiment, the step of removing the complexes is accomplished by removing the fluid within the target region with a removal device. For example the removal device can be a syringe or cannula. In another example, the fluid is blood and the flow of blood through the target region is temporarily halted during the removal of the complexes.

In a further embodiment of the method, the disease-associated circulating entities are a first population of cells of the organism, and the first target surface structure on the first population of cells distinguishes the first population of cells from other populations of cells in the organism. In yet another embodiment, the first disease-associated circulating entity comprises a plurality of different target surface structures and the nanoparticles are functionalized so as to comprise a plurality of different bioaffinity molecules, wherein each different bioaffinity molecule binds specifically to one of the plurality of different target surface structures on the first disease-associated circulating entity.

In a further embodiment of the method, the disease-associated circulating entities are a first population of cells of the organism, and the first target surface structure on the first population of cells distinguishes the first population of cells from other populations of cells in the organism. In yet another embodiment, the first disease-associated circulating entity comprises a plurality of different target surface structures and the nanoparticles are functionalized so as to comprise a plurality of different bioaffinity molecules, wherein each different bioaffinity molecule binds specifically to one of the plurality of different target surface structures on the first disease-associated circulating entity. In yet another embodiment, the population of disease-associated circulating entities comprises a plurality of different target surface structures and the nanoparticles are functionalized so as to comprise a plurality of different bioaffinity molecules, wherein each different bioaffinity molecule binds specifically to one of the plurality of different target surface structures on the disease-associated circulating entities.

In one aspect the invention comprises fNPs that comprise the following properties:
a. paramagnetic or superparamagnetic
b. compatible with in vivo use
c. functionalization to bind specific DACEs
d. can be immobilized through the use of a magnetic field

In one aspect, the invention is a therapeutic system comprising one or more populations of paramagnetic or superparamagnetic nanoparticles that each contain at least one functionalization with a bioaffinity molecule, whereby the bioaffinity molecule is chosen to target a specific Disease, and wherein the bioaffinity molecule is capable of binding to a bioaffinity target on a DACE, introducing such populations of such fNPs into a part of an organism, in particular into a biofluid of the organism, allowing or enabling such fNPs to form complexes with DACEs that may be present in said part of the organism, and removing such complexes from the organism.

In one particular aspect of the invention the part of the organism into which the nanoparticles are introduced is the circulatory system, the lymphatic system, or a body cavity of a patient, such as the peritoneum, a lung, the bladder, the digestive tract, or the colon. This part of the organism is also referred to as Body part.

In one particular aspect the invention relates to removing pathogenic cells from the circulation or lymphatic system or body cavity by magnetic force.

The invention further relates to methods to enhance the initial association and complex formation of the nanoparticles with the DACE. These methods enhance the kinetics of binding by applying a magnetic force to the superparamagnetic or paramagnetic nanoparticles, resulting in their relative motion with respect to the surrounding fluid that contains the DACEs.

The invention also relates to methods of removing such particle-associated DACEs from the body. fNPs that bind to DACEs in vivo are arrested by magnetic force and removed from circulation. The arrest embodies the capture of DACEs that have taken up some of the introduced functionalized nanoparticles by the use of a magnetic field.

In one embodiment, a magnetic capture device is applied externally to a vein of the subject to retain (“arrest”) the particles and their associated DACEs in a defined location or volume. In another embodiment, the magnetic capture device is inserted into the body to arrest the fNP-DACE complexes in vivo.

In one particular embodiment, the DACEs in the circulatory system are removed by introducing fNPs into the circulatory system by injection, the formation of fNP-DACE complexes during circulation in the body, concentration or retention of the complexes in a target site and removal or destruction of the complexes. In one embodiment, the removal comprises venipuncture or a blood draw of the blood volume containing the fNP-DACE complexes at the site of arrest. In another embodiment, the complexes are removed from the lymphatic system by incubation to facilitate the formation of fNP-DACE complexes in the circulatory or lymphatic system and subsequent removal of the resulting fNP-DACE complexes from the lymphatic system, for example by removing of the lymph node in which the particles have been captured.

One embodiment includes introducing fNPs into the circulatory system of a subject where fNP-DACE complexes form through specific interactions between the nanoparticle functionalization and the complementary DACE epitope or target entity. The complex formation can optionally be enhanced through methods, such as magnetic mixing, that increase mixing of the particles and cells in the circulatory system. Next, magnetic fields generated by a magnetic capture device are applied to collect the fNPs and their associated DACEs in a defined location, the target region in the body, e.g. a section of a vein by applying a magnet to the area. Ultimately, the fNPs and associated DACEs are removed from the body, through a method such as venipuncture.

The invention draws knowledge about suitable Bioaffinity molecules and Bioaffinity targets from an extensive research into the biology of disease and immune-system associated cell and biological entities. It further builds on the research into disease diagnostic and immunomagnetic methods for identification of superior fNPs. The invention further benefits from experience with in vivo use of nanoparticle as used in some ablation and theragnosic approaches and from many years of experience with nanoparticles in in vivo imaging approaches. The invention adds to the arsenal of local and systemic methods to combat disease. It's novel and minimally invasive approach of the removal of DACEs may surpass other systemic methods as it prevents the damage to desirable cells and makes the treatment accessible to patients even outside of highly specialized and well equipped treatment centers.

The advantages of the present invention include preventing or delaying cancer metastasis by removing CTCs from circulation. Metastases, seeded from CTCs, are responsible for ˜90% of mortality of cancer patients. Successful removal of CTC circulation would add a significant new option for intervention of cancer progression and supplement other therapeutic options.

### BRIEF DESCRIPTION OF THE DRAWINGS

FIG. 1: Magnetic capture device: Assembly of individual magnetic blocks that make up a linear (1D) Halbach array and simulated magnetic field lines as generated by the array. The solid arrows inside the blocks indicate the magnetic flux orientation (arrow tips=north) inside of each individual magnetic block. The Halbach-type orientation of the blocks results in a relative amplification of the magnetic field above the array. The magnetic capture device is positioned relative to the body so that the target region in which nanoparticle concentration and capture occurs is located within the magnetic field above the array. Modified from Wikimedia Commons, [CC-BY-SA-3.0 (http://creativecommons.org/licenses/by-sa/3.0)], via Wikimedia Commons.

FIG. 2: Magnetic capture device: Assembly consisting of five cubical magnets arranged in a cross-like pattern to form a two-dimensional (2D) Halbach array. The magnetic field of the center magnet is oriented vertically upwards (circle with center dot=north) and perpendicular to all other magnetic field orientations. The magnetic fields of the four outward magnets point radially towards the center magnet as indicated by the arrows (“X”=south). In this orientation, magnetic field strength and gradient are amplified above the assembly and centered over the circle with center dot.

FIG. 3: Magnetic capture device: Assembly consisting of eleven cubical magnets arranged in two cross-like patterns, with the two center magnets being oriented vertically up (circle with center dot) another one down (“X”).

FIG. 4: Magnetic capture device: Assembly using two layers of 2D Halbach arrays as described in FIGS. 2 & 3. The magnetic field strength is increased by adding layers of magnets in the same orientation.

FIG. 5: Magnetic capture device: Examples of three-dimensional (3D) assemblies that are stacked and further integrated to increase nanoparticle capture efficiency across the target region.

FIG. 6: Top: Size distribution of a superparamagnetic ironoxide nanoparticle as measured by diffuse light scattering showing that 3 batches of particles produce consistently nanoparticles of 50 nm+−10 nm size. Bottom: Fluorescence spectrum of two concentrations of nanoparticles fNP-2 functionalized with human-EpCAM and Phycoerythrin (PE).

FIG. 7: Microscopy images with bright filed illumination (left) and fluorescent lighting (right) for HCT-116 (top) and MCF-7 (bottom). HCT-116 show little aggregation in the white light image and clear staining with the fluorescent antibody. Also apparent is the recruitment of EpCAM in the interface of cell-cell interaction.

FIG. 8: Fluorescence images (presented inverted and in grey-scale) showing cell lines (BXPC3, HCT-116, SU8686 and PaNC1) as marked. In each figure the location of the cell nuclei marked by DAPI appears as larger, uniform light grey areas. The (EpCAM-PE-functionalized) nanoparticles (fNP-2) are visible as clouds of small dark-grey dots. Arrows indicate fluorescence signal generated by phycoerythrin (PE) fluorescent antibodies against EpCAM antigens that are present on the surface of the targeted cells BXPC3, HCT-166 and in particular in the space between adjacent cells. SU8686 and PANC1 cells show little staining by the fluorescent-marked nanoparticles, indicating little to no complex formation.

FIG. 9: Scatter diagrams of HCT-116 captured by fNPs and triple stained with h-EpCAM antibody, CD-45 antibody and propidium iodide (PI). Top row: FACS forward vs. side scatter (left), Forward vs. wide scatter (center), CD45-APC vs. EpCAM-FTIC (right). The cell population selected in P2 corresponds to non-aggregated, live cells, as determined by minimal scatter (P1) and PI stain (P2). The remaining scatter diagrams in the figure show the scatter diagrams of CD45 vs. EpCAM staining for various samples and controls, specifically top right: Cells after isolation from PBS buffer containing 100,000 cells/ml. Center row left to right: cells isolated from whole blood spiked with 100,000 cells/ml, 10,000 cells/ml, and 1000 cells/ml, respectively. Bottom row, left to right, cells isolated from buffer spiked with 1000 cells/ml, cells isolated from whole blood spiked with 100,000 cells/ml using a control fNP (anti-biotin), whole blood without spiked cells.

FIG. 10: Absolute cell count after spiking of about 100,000 cells into 1 ml of blood. fNP-1 effectively captures more than 70% of the 100,000 cells spiked into blood. fNP-2 is considerable less effective.

FIG. 11: Left panel: Laser scatter of an unlabeled nanoparticle suspension before injection in a FACS flow cytometer. Center panel: Laser scatter of the injected nanoparticle suspension after being recaptured by magnetic force from the tail of a mouse after three hours of circulation, i.e. from the drop of blood that was retrieved from the tail tip and washed 3 times to remove blood components. Right panel: Control laser scatter of regular blood components (e.g. red blood cells).

### DETAILED DESCRIPTION OF THE INVENTION

### Magnetic Capture Device

The magnetic capture device may comprise a permanent magnet or an electromagnet. The magnetic capture device is capable of generating a magnetic field and field gradient sufficient to magnetize paramagnetic or superparamagnetic nanoparticles. A magnetic field gradient is required to exert a translational force on a magnetic dipole. The magnetic capture device may also comprise other ferromagnetic materials with a high permeability that amplify magnetic flux density and magnetic field gradient in the target region for more efficient capture.

To allow for the concentration and capture of nanoparticles from a biofluid, the magnetic capture device generates a high magnetic flux density and a large magnetic field gradient that reaches sufficiently far into the target region where the nanoparticles are to be collected. The magnetic flux density in the target region should be at least 0.1 T, preferably at least 0.5 T, and more preferably 1 T or greater. The magnetic field gradient in the target region should be at least 100 T/m, preferably at least 1,000 T/m, more preferably 10,000 T/m or greater, and most preferably 105 T/m or greater. The distance of the magnetic field reaching from the magnetic capture device into the target region as determined by the 1/e value of magnetic flux density compared to the maximum value at the magnetic capture device should be at least 0.3 mm, preferably at least 1 mm, more preferably 3 mm or greater, and most preferably 10 mm or greater.

The magnetic capture device will be capable of generating a magnetic field strong enough to concentrate and capture DACE-NP complexes in a biofluid. The magnetic capture device may also be capable of generating a magnetic field strong enough to capture uncomplexed nanoparticles from a biofluid. A typical magnetic capture device as used here is comprised of an array of individual N45 NdFeB block magnets with an energy product of 45 MGOe (358 kJ/m3) and a Curie temperature of about 80 C.

### 1) External Magnetic Capture Device

In one embodiment the magnetic capture device comprises an externally applied magnet that effects the concentration and immobilization of nanoparticles in the target region. The concentration and capture takes place inside the body with the magnetic field acting from the outside through the body of the patient. Nanoparticles are immobilized in a subvolume of the target region that is located in closest proximity to the magnet capture device. An example of a target region, without limitation, is a defined section of a vein through which blood flows. The magnetic capture device is placed directly onto the target region to effect the concentration and capture through the body of a patient.

In this embodiment, the magnetic capture device typically is placed as closely as possible to the area in which the fNPs are to be collected, with a set of magnets that form a configuration or array to generate a magnetic field gradient that reaches into the target region.

Methods of configuring individual magnets to generate such field gradients may comprise individual magnets that are placed or generated in situ in close proximity or next to each other such that their magnetic field orientation differs from its nearest neighbor(s), thereby creating magnetic field lines of a small radius of curvature.

Examples of such arrays are magnetic cubes arranged next to each other with alternating magnetic direction (i.e. north/south/north . . . etc.), in either a one-dimensional (linear) or two-dimensional (checkerboard) assembly, as described for instance in⁶⁶.

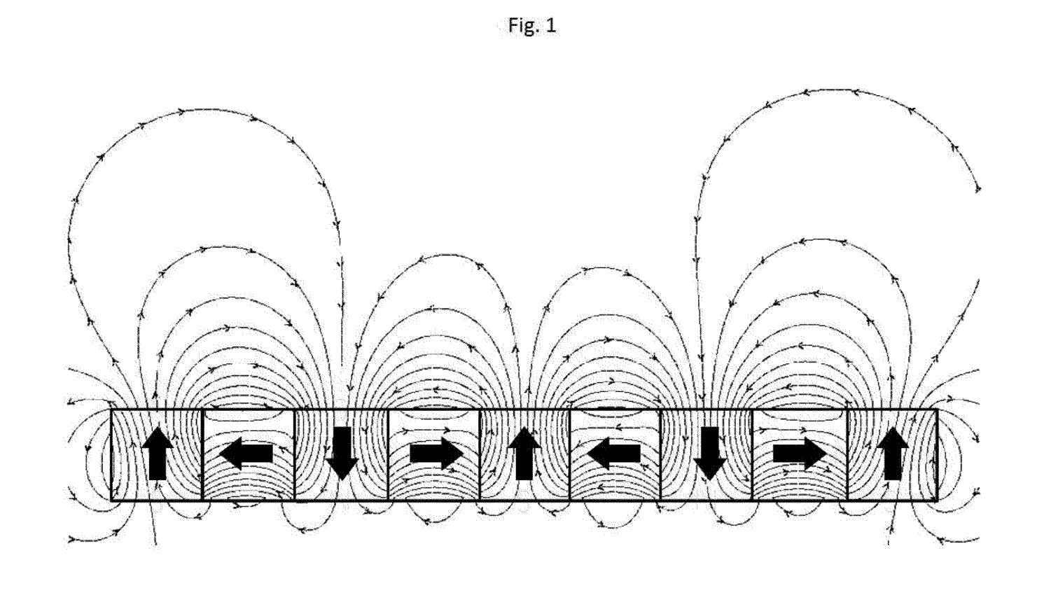

In another embodiment, the magnetic capture device may comprise further assemblies of individual magnets that are designed to produce an enhanced magnetic field and field gradient on one side, which is the side oriented towards the target region for nanoparticle capture⁶⁷. Examples of such magnetic structures are Halbach arrays, shown in FIG. 1, or flexible magnets with embedded, alternating magnetization commonly referred to as refrigerator magnets.

In another embodiment, the magnetic capture device may comprise two- and three-dimensional magnet assemblies that increase nanoparticle capture efficiency by further concentrating field gradients and amplifying the magnetic flux density generated by the magnetic capture device across the target region.

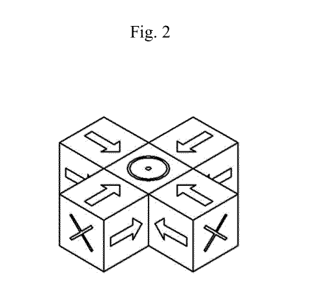

The simplest such embodiment is an assembly consisting of five cubical magnets arranged in a cross-like pattern, where the four outward magnets are oriented pointing radially towards the center magnet, with the center magnet oriented vertically and perpendicular to all other magnet orientations (circle with center dot) as shown in FIG. 2.

In a preferred embodiment, the dimensions of each of the magnetic cubes that form the assembly are 1 mm×1 mm×1 mm. In another preferred embodiment, the dimensions of each of the magnetic cubes that form the assembly are 2 mm×2 mm×2 mm. In yet another preferred embodiment, the dimensions of each of the magnetic cubes that form the assembly are 3 mm×3 mm×3 mm.

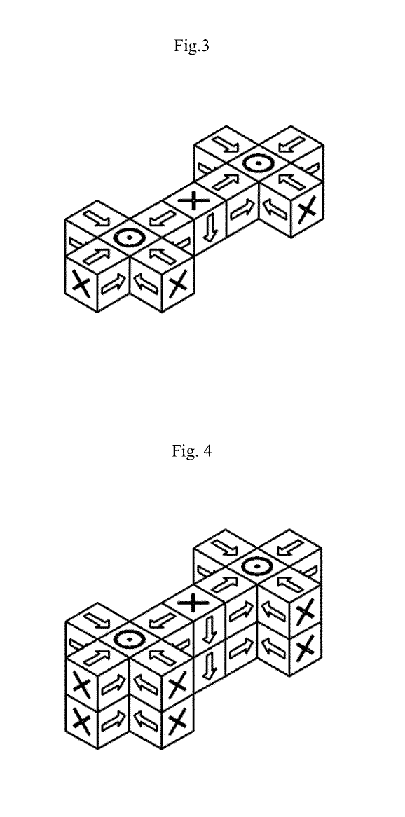

The assembly of FIG. 2 can be further combined for example into a structure consisting of eleven magnets arranged in two cross-like patterns, with the two center magnets being oriented vertically up and a connecting magnetic cube in opposite direction to the two centers as shown in FIG. 3. In this orientation, the field strength is amplified above the array and centered over the circle with center dot. The magnetic capture device is positioned relative to the body so that the target region in which nanoparticle concentration and capture occurs is located within the magnetic field above the array.

The field strength of these assemblies can be further increased by adding additional layers of magnets in the same orientation, as shown in FIG. 4. It is readily apparent that identical or essentially similar field distributions can be achieved with other shapes of magnetic material, such as non-cubical blocks, disks, cylinders, rods, triangles, pyramids, spheres, or ovals.

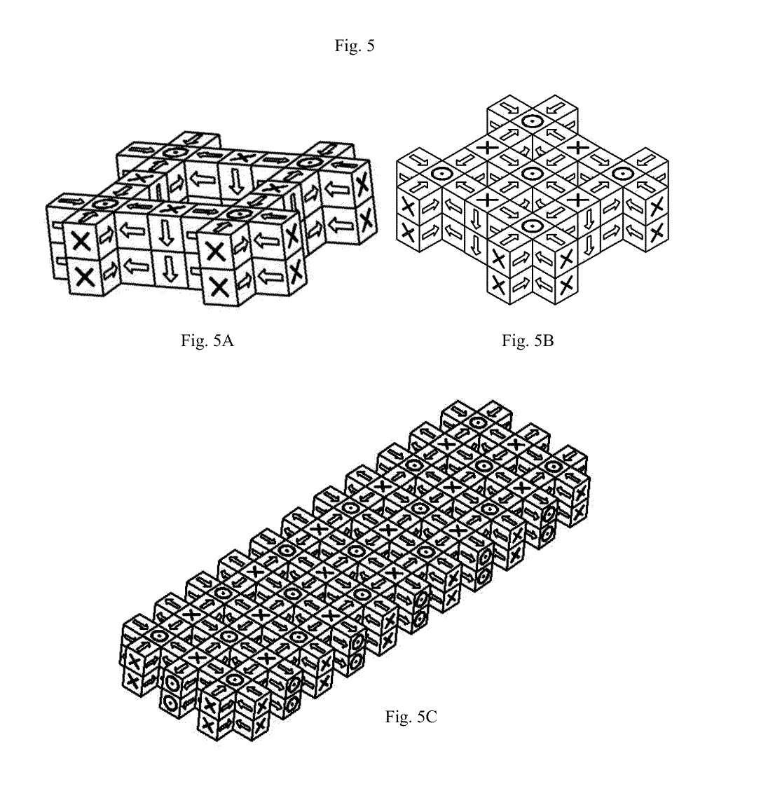

In another preferred embodiment, the magnetic capture device may comprise two- and three-dimensional magnet assemblies that are stacked or further integrated to increase nanoparticle capture efficiency across the target region. Examples for various configurations of such Halbach-type assemblies are shown in FIG. 5. The dimensions of an assembly are selected to maximize overlap of the magnetic field and field gradients generated by the assembly with the target region. For example if using the array to concentrate and capture nanoparticles in a vein, the array shown in FIG. 5c is aligned with the orientation of the vein.

Further embodiments may also comprise magnets that are arranged diagonally⁶⁷ or at various other angles, as well as assemblies of magnetizable materials that may exhibit curved internal magnetizations as generated by the application of external magnetic fields with high gradients and high field strength during production.

Application of the magnetic capture device and removal of nanoparticles from the body

In a preferred embodiment, the magnetic capture device is applied directly to the target area by a suitable fastener, such as a bandage, clip, belt, sleeve, an adhesive strip, or a combination thereof. The application is done such that the magnetic assembly is placed directly over the target area, aligning as necessary with the targeted internal body structure or cavity. For example when collecting nanoparticles from the blood flow in a vein (such as the antecubital (or cephalic), radial, ulnar, brachial or subclavian vein), the magnet assembly will preferably also have a linear structure (as depicted for instance in FIGS. 1, 5c) which will be aligned with the direction of the blood flow so as to maximize the overlap of the magnetic field with the target region and thereby the efficiency of capture.

The application of the magnetic capture device will last until the desired amount of nanoparticle has been immobilized from the target region. Preferred application times are between 20-30 minutes, between 30-60 minutes, and between 60-120 minutes.

After the immobilization of the nanoparticles has occurred, the target region is stabilized to avoid loss of the nanoparticles from the target region during removal from the body. Examples for stabilizing the target region prior to removal of the nanoparticles are through the use of a tourniquet or by applying suitable pressure to the vein above and below the magnetic capture device so as to temporarily prevent blood from circulating through the target region. The target region is then accessed with a hypodermic needle for removal while the magnetic capture device is removed to release the nanoparticles. The suspension and removal of nanoparticles is aided by gently massaging the target region after the removal of the magnetic capture device.

Alternatively the concentration, capture and subsequent removal of the nanoparticles may occur by making use of a central line that may be placed or may already have been placed in the patient for other purposes of intravenous access. This type of access to the target region is preferable because a large bore cannula may be easily placed.

Alternatively—in particular for target regions other than the circulatory or lymphatic system—access to the target region for the removal of the immobilized nanoparticles may occur through a suitable small surgical incision.

### 2) Internal Magnetic Capture Device

In another embodiment the magnetic capture device comprises an internally applied magnet that effects the concentration and immobilization of nanoparticles in the target region. The concentration and capture takes place inside the body with the magnetic field acting from the inside through the body of the patient. Nanoparticles are immobilized in a subvolume of the target region that is located in closest proximity to the magnet capture device, or on the magnet capture device itself.

In a preferred embodiment, the magnetic capture device is temporarily inserted into the patient by making use of a central line with a large bore cannula. This type of access to the target region allows the insertion of sub-mm magnetic assemblies. In a preferred embodiment, the magnetic capture device is a thin structure that comprises one-dimensional or multidimensional magnetic capture assemblies⁶⁸.

In a preferred embodiment, the magnetic capture device for internal capture is connected to a flexible line or lead for the purpose of placing it into and retrieving it from the body. In a preferred embodiment, the flexible line is made from a flexible material (e.g. nylon), coated preferably with a material such as Teflon, PEG or other low-immunogenicity materials as known in the medical device field.)

In a preferred embodiment, the magnetic capture device is coated with an anticoagulant to prevent the formation of blood clots during treatment.

In another embodiment, the magnetic capture device as used internally may comprise two or more subassembly structures that are arranged in a sandwiched fashion so as to create magnetic field gradients throughout a volume between the subassembly structures that is accessible by fluid flow and in which the nanoparticles are captured from the biofluid. The principal design of such subassembly structures, albeit at larger dimensions as for the present invention, is illustrated for instance in U.S. Pat. No. 7,161,451 FIGS. 1-4, which describes quadrupolar and hexapolar arrangements of magnetic blocks with alternating opposite field orientations and an accessible volume between two subassembly structures.

In another embodiment, the magnetic capture device contains an electromagnet. The electromagnet can be used to indicate the presence of magnetizable materials such as fNPs or complexed fNPs that are being captured by it.

### Magnetic Mixing

In order to achieve efficient capture and removal of DACEs it is advantageous to facilitate fast on-rates as well as high selectivity and efficiency of binding of the functionalized particles to the targeted cells. Due to the small diffusion coefficient of nanoparticles—such as DACEs and nanoparticles—the rate and efficiency of binding DACEs to the particles is significantly reduced compared to the binding of smaller molecules that have comparable affinity but which are able to readily diffuse in solution.

Relative motion between the targeted cells and the functionalized particles overcomes this problem. Relative motion can be achieved by different means, such as by moving the particles used for capture back and forth through the solution by the application of magnetic fields, by repeated precipitation and suspension, by shear-flow conditions generated by the passage of both fluid (i.e. blood plasma) and solid components (i.e. cells and functionalized particles) through narrow capillaries, or by electrophoretically generated movement. Magnetic fields can act on particles that are magnetic or magnetizable or carry a net charge.

Magnetically generated relative motion can be achieved in several different ways: In a preferred embodiment, the motion of the particles as part of the flow of fluid in the circulatory system itself is used in conjunction magnetic fields that are applied from the outside of the body. The magnetic field causes relative motion of the circulating paramagnetic or superparamagnetic nanoparticles with respect to the surrounding fluid and DACEs but is insufficient to immobilize the particles.

A magnetic mixing device used for this purpose preferably has an array of multiple magnets arranged in a spatially varying magnetic pattern along a volume of the body wherein the particles are to be mixed. For example if the magnetic mixing is to be achieved in the blood flow of a vein, magnetic mixing device preferably has magnets arranged in alternating orientation along the direction of the vein. It is preferable for the magnetic field to have a gradient that reaches sufficiently far into the target region where the nanoparticles are to be mixed, but the field gradient is considerably smaller than in those fields used to capture and fully immobilize the nanoparticles. The magnetic field gradient in the magnetic mixing region preferably is about 0.1-1 T/m, more preferably 1-10 T/m, and most preferably 10-100 T/m.

The magnetic field generated by the magnets used for magnetic mixing overlaps the magnetic mixing region. The degree of overlap is defined as the distance from the magnetic mixing device at which the nanoparticles are exposed to at least 1/eth of the maximum magnetic flux density that is generated by the magnetic mixing device. For applications of mixing particles in a bloodstream close to the skin, such as in a vein, the overlap preferably is 1-3 mm, more preferably 3-5 mm, and most preferably 5-10 mm. For applications of magnetic mixing with magnetic mixing regions situated deeper in the body the overlap preferably is 10-20 mm, more preferably 20-30 mm, and most preferably 30-300 mm.

Magnetic capture devices as described above can serve as magnetic mixing devices if they are placed such that they do not fully immobilize the nanoparticles. In a preferred embodiment the distance of the magnetic device to the body is changed such that in one position of close proximity or contact with the body the device immobilizes the particles, whereas in a second position further distant from the body the device results in relative motion but no immobilization of the nanoparticles. A preferred distance between position one and position two is at least twice of the distance at which the magnetic flux density reaches 1/eth of the maximum magnetic flux density that is generated by the magnetic device.

Examples of other magnetic arrays comprised of static magnets with alternating orientation that can be used for magnetic mixing are those that are often used in the alternative medicine praxis of magnet therapy. While no effect of these magnets has been found on the endogenous system, the fields they create can be strong enough to generate motion of nanoparticles relative to non-magnetic endogenous DACEs. Simple polymer-embedded magnetic sheets (“refrigerator magnets”) as well as other devices commonly used for the collection of magnetic particles from multiple tubes or reaction vessels are other examples. Multiple magnetic sources that are typically combined in a pattern or array format. For applications outside the circulatory system where sufficient surface accessible locations are not available to allow such a field to be applied, suitable other sources of magnetic field changes may be needed. In one embodiment, an MRI instrument can be employed to generate fluctuating magnetic fields to enhance complex formation between fNPs and DACEs.

The distance of the magnetic device to the collection point (i.e. preferably a vein directly underneath the skin of an easily accessible area of the body, such as wrist or elbow) can be adjusted so that the magnetic field gradient generates either only a mixing action (with the magnetic device removed from direct skin contact and separated from the collection point between 5-50 mm, or up to 100 mm) or a collection of particles under the magnet (typically with the magnetic device being either in direct skin contact or in close proximity, typically being separated from the collection point between 0-5 mm, and up to 20 mm).

Another version is the use of alternating magnetic fields so as to induce a relative motion regardless of whether the magnetic particles are at rest or in flow. This embodiment requires the use of either movable static magnetic fields (permanent magnets are mechanically moved over the fluid containing the particles and thereby create relative motion in the fluid) or by electrically alternating magnetic fields in a static setup, or a combination of either one of these embodiments.

### Disease-Associated Circulating Entity

The term Disease-Associated Circulating Entity (DACE) shall refer to any particle, material, chemical or biological agent or organism that is desired to be selectively removed from an organism. A DACE may be, for example, any unicellular or multicellular organism (e.g. bacteria, virus, fungus, parasite), certain types of blood cells (e.g., autoreactive T-cells, B-cells), any subset of leukocytes cancer cells such as CTCs or cancer stem cells, any cell or organism circulating in the blood of a higher organism, specific molecules, proteins, antibodies, antigens, chemicals, as well as inflammatory, immunogenic or plaque-forming agents, or any combination thereof.

We define Body Fluid as any fluid component in an organism that may carry a DACE including the body fluids found in the circulatory (i.e. blood), lymphatic or ventricular system, biofluids from body cavities such as the peritoneal cavity or the digestive system, or the central nervous system of a higher organism, including but not limited to humans.

Without limitation, an example of DACEs in the field of virology are viruses and virus-infected cells. For example, the viral load and the viral composition (the quasi-species distribution) of human immunodeficiency viruses HIV is indicative of disease progression and corresponding selection of treatment options. The reduction in viral load in the blood is one measure of the efficacy of therapy. Endogenous cells infected by a virus also become DACEs.

Bacteremia, the presence of bacteria in the blood, can induce a severe immune response, leading to septic shock. Bacteria also frequently spread through the blood to different parts of the body where they cause infections and secondary diseases. Bacteria in the blood are thereby a DACE and its removal would produce a beneficial effect.

In the field of immunology, many diseases are associated with the presence of specific immune cells or antibodies or cytokines that cause inflammatory responses, which, when uncontrolled, cause disease. Reduction of such inflammatory agents can reduce the unwanted inflammatory response. There are over one hundred accepted autoimmune diseases, representing severe unmet medical need and the removal of DACEs can help address those needs.

Diseases of the cardiovascular system and metabolic diseases are in many cases associated with the presence of DACEs in circulation. For example, arterial deposits are causing cardiovascular disease. Reduction of plaque-associated components, e.g. certain macrophages, and endogenous particles such as low-density lipoproteins can have beneficial effects to the health of a patient.

In the field of oncology, CTCs are one example of DACEs. CTCs are cells that originate from a tumor and have acquired the ability to enter the circulation. CTCs are associated with poor prognosis for the cancer patients. CTCs are used herein as an example to illustrate the individual and public health benefits of the method. CTCs are exfoliated from solid tumors and have been found in very low concentrations in the circulation of patients with advanced cancers of the breast, colon, liver, ovary, prostate, and lung, and the presence or relative number of these cells in blood has been correlated with overall prognosis and response to therapy. These CTCs may be an early indicator of tumor expansion or metastasis before the appearance of clinical symptoms

### Bioaffinity Molecules

Bioaffinity molecules can be one or more of several types of protein, peptide, nucleic acid, antibody, antigen or ligand or hapten that are attached to the Nanoparticle and that are selected to bind to a bioaffinity target on the DACEs.

Bioaffinity targets are three-dimensional structures that are specific epitopes for a DACE. If the DACE is a non-cellular target, such as a small molecule or a biological molecule (for example, protein, oligonucleotide, lipoprotein, glycoprotein, small particle, vesicle) the bioaffinity target is a epitope or active site or allosteric binding site or a hydrophobic surface patch or a specific nucleic acid sequence that can be complemented, or a exposed three-dimensional arrangement of a part of the DACE surface. If the DACE is a cell or cell-like assembly the bioaffinity target is preferably one or more surface epitopes or other exposed components on a DACE. The bioaffinity target that is recognized by the bioaffinity molecule is at least partially exposed on the surface of the DACE. DACE-specific surface molecules may be specific receptors, or cell specific surface proteins, or exposed glycoproteins, or combinations thereof. For example a naturally occurring cell-surface receptor on the DACE would be such a surface epitope. Surface epitopes are preferably such that they are expressed specifically on target cells. For example, EpCAM is a cell surface protein that is associated with circulating tumor cells. EpCAM is not found on cells in circulation of healthy individuals.

The bioaffinity molecules and bioaffinity targets represent a pair of surface structures, typically molecules that form a high affinity bond between each other. The pair of bioaffinity molecule and bioaffinity target is chosen as to form a specific binding pair to allow for selective pairing of a Nanoparticle and a DACE. Such binding pairs typically referred to as “ligand/ligate” binding or interaction and are exemplified by, but not limited to, antibody/antigen, antibody/hapten, enzyme/substrate, enzyme/inhibitor, enzyme/cofactor, binding protein/substrate, carrier protein/substrate, lectin/carbohydrate, receptor/hormone, nucleic acid/nucleic acid, oligonucleotides/nucleic acid, receptor/effector or repressor/inducer bindings or interactions.

Methods to identify and select suitable bioaffinity molecule/target pairs and to determine the quality of complex formation well known standard biochemical techniques. In a preferred embodiment a method to identify such pairs is the creation of a monoclonal or polyclonal antibody against a DACE. In another preferred embodiment, biochemical screens known in the art, (e.g. a two-hybrid screen, used in enzymology) can be used to find identify and characterize enzyme/inhibitor pairs or receptor/ligand or protein-protein interaction pairs.

Without limitation a bioaffinity molecule may comprise an antibody or antibody-like molecule, including bifunctional antibodies, or any structure that binds specifically to DACEs, like a hapten or drug or carbohydrate or a peptide or a protein (where protein is, a naturally expressed or disease associated protein, including modified (e.g. glycosylated) proteins. bioaffinity molecules that are haptens may comprise peptides or small molecules of or proteins that specifically bind to DACE-specific surface molecules.

In a preferred embodiment that bioaffinity molecule is an antibody and the bioaffinity target is a complementary antigen.

In one embodiment, the bioaffinity molecule functionalization of the nanoparticles to bind the bioaffinity target on the DACEs is an Antibody-like Molecule. An antibody-like molecule is an antibody or a fragment thereof, such as the Fab fragment, or a mimic thereof, which may comprise any protein that has been designed or selected or evolved to bind an antigen or hapten.

Binding in this context means a high affinity attachment, non-covalent or covalent, such as an intermolecular interaction, preferably with a dissociation constant (K_{d}) of less than 10 exp(−9) M (molar). Designs wherein there are more than one binding pairs (e.g. multifunctional antibodies, multiple binding interactions between within an fNP-DACE complex), the overall dissociation constant of all binding interaction is of relevance.

Antigens can be peptides, proteins, polysaccharides, saccharides, lipids, nucleic acids, or combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof.

In one particular embodiment the Antibody-like Molecule may comprise an affinity for EpCAM (also referred to in the literature as CD326), an epithelial cell marker commonly found on circulating tumor cells, but rarely found on other cells in the circulation of healthy individuals. For example the Antibody-like molecule might be an antibody against human EpCAM. EPCAM accession number is NM 002354. Its Entrez gene ID is 4072.

In an alternative embodiment such bioaffinity molecule is directed against a cancer stem cell (CSC) marker. In one embodiment, such CSC marker is a chemokine receptor such as CXCR4, or CXCR7.

In yet another embodiment, such bioaffinity molecule is directed against epithelial proteins being preferably cytokeratin 8, cytokeratin 18 and/or cytokeratin 19.

In yet another embodiment, such bioaffinity molecule is directed against MUC1.

In yet another embodiment, such bioaffinity molecule is directed against a protein regulating the cell proliferation, such as Her2, EGFR, IGFR, or any other receptor-tyrosine kinase.

Many suitable antibody-like molecules are commercially available and their specificity for disease target may be described in the literature. Such antibody-like molecules can, for example, be generated by methods know in the art to produce either monoclonal or polyclonal antibodies, or recombinant techniques. Antigens, useful for generating such antibodies are also known in the art or are available from public or private or commercial resources. Antigen may be also be a whole active, inactivated or attenuated DACE or a part thereof that is presented at the surface of the DACE, such as. For example, a receptor fragment, surface oligosaccharide, any protein or fragment thereof expressed on the surface of the cell.

The antigens can comprise recombinant polypeptides produced by expressing DNA encoding the polypeptide antigen in a heterologous expression system. The antigens can comprise DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

In one embodiment the binding partner attached to the nanoparticle may be an antibody or antibody fragment recognizing a tumor antigen.

Antigens may also be small molecules and small molecules presented on a suitable carrier to increase the immunogenic potential.

The binding partner attached to the nanoparticles can be a ligand recognized by cell-specific receptors. For example, neuraminic acid or sialyl Lewis X can be attached to a superparamagnetic nanoparticle. Such conjugates are suitable for the treatment or prophylaxis of diseases in which bacterial or viral infections, inflammatory processes or metastasizing tumors are involved. Ligands to molecular receptors are preferred embodiments to act as bioaffinity molecules. Those ligands are naturally occurring or exogenous molecules that attach with high affinity and good specificity to molecules on the DACE. For example, ligands may comprise molecules for tyrosine-kinase receptors such as EGFR, or Her2, etc., or ligands for receptors that are typically found on specific cells of the immune system, such as ligands for cytokines, CD40, the T-cell receptor, chemokines, GPCRs, drug transporters, etc.

Other ligands, such as protein or synthetic molecules that are recognized by receptors can be associated with the superparamagnetic nanoparticles. In addition the binding partner attached to or associated with the superparamagnetic nanoparticles may be a peptide, DNA and/or RNA recognition sequence.

The term “aptamers” as used herein refers to nucleic acids (typically DNA, RNA or oligonucleotides) or peptides that bind to a specific target molecule. Methods for making and modifying aptamers, and assaying the binding of an aptamer to a target molecule are known to those of skill in the art (see for example, U.S. Pat. Nos. 6,111,095, 5,861,501 and others). Ligands that bind aptamers include but are not limited to small molecules, peptides, proteins, carbohydrates, hormones, sugar, metabolic byproducts and toxins. Aptamers configured to bind to specific target can be selected, for example, by synthesizing an initial heterogeneous population of oligonucleotides, and then selecting oligonucleotides within the population that bind tightly to a particular target molecule. Once an aptamer that binds to a particular target molecule has been identified, it can be replicated using a variety of techniques known in biological and other arts, for example, by cloning and polymerase chain reaction (PCR) amplification followed by transcription.

The target cell may contain one or more binding partners on its surface. Binding partners that may be on the surface of the cells include, but are not limited to, cancer antigens, viral antigens, bacterial antigens, protozoan antigens, and fungal antigens.

In one embodiment, the bioaffinity molecule are molecules that binds to proteins on the surface of cancer cells and that are largely absent on the surface of non-cancer cells. Exemplary cancer specific proteins include, but are not limited to cancer antigens also referred to as tumor specific antigens or biomarkers. An increasingly large body of literature is available to disclose such biomarkers.

In one particular embodiment, this invention enables the validation and characterization of such biomarkers. The depletion of a DACE comprising such biomarkers can establish if the marker or the DACE carrying the specific a marker is causative or a suitable target to combat the disease. Such an approach is commonly referred to as biomarker validation and has utility in identifying suitable targets for diagnostic purposes in addition to having direct therapeutic interest.

Other affinity molecules on the nanoparticles are those that bind to the transferrin receptor, MUC1, one or more ErbB receptor or any other growth factor receptor that is found present or strongly overexpressed on cancer cells. Cell surface proteins including PSA, TACE, MMP-14, CEA (carcinoembryonic antigen widely overexpressed in a wide variety of cells), Urokinase receptor (overexpression is strongly correlated with poor prognosis in a variety of malignant tumors) and CXCR4 (linked to breast cancer invasion and metastasis), immune system markers such as CD3, CD2, Fc gamma R activating receptor (CD16), glycosyltransferase-1,4-N-acetylgalactosaminyltransferases (GalNAc), melanoma antigen gp75; human cytokeratin 8; high molecular weight melanoma antigen, overexpressed products of neu, ras, trk, and kit genes, mutated forms of growth factor receptors or receptor-like cell surface molecules (e.g., surface receptor encoded by the c-erb B gene), the tumor associated antigen, mesothelin, defined by reactivity with monoclonal antibody K-1, is present on a majority of squamous cell carcinomas including epithelial ovarian, cervical, and esophageal tumors, and on mesotheliomas.

Tumor antigens of known structure and having a known or described function include the following cell surface receptors: HER1 (GenBank Accession No. U48722), HER21994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Ace, Nos. X51730, X69068 and M15716), follicle stimulating hormone receptor (FSH-R) (GenBank Acc. Nos. 234260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Acc. No. 573003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. 302289 and J02038); p97 (melanotransferrin) (Brown, et al., J. Immunol., 127:539-46 (1981); Rose, et al., Proc. Natl. Acad. Sci. USA, 83:1261-61 (1986)). In addition, cancer specific epitopes, including bladder cancer specific epitopes (U.S. Pat. No. 2012/0230994. Other tumor-associated and tumor-specific antigens are known to those of skill in the art and are suitable for targeting using the disclosed nanoparticles.

A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxyiridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chrornatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

Antigens of parasites can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosomai mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

Other Molecules that can act as a DACE and comprise a bioaffinity target include xenobiotics, endogenous metabolites, toxins, circulating DNA, RNA, including mRNA, and any type of regulatory nucleic acid that is found outside a cell, proteins and protein complexes, peptides, and peptide assemblies, including those found in plaques, tangles, and any other disease-associated molecular assemblies. These bioaffinity targets may be isolated within a biofluid or be part of molecular assemblies, complexes or similar. In one embodiment, phosphor-tau protein, a hyperphosphorylated form of the tau protein found in the CNS can be a DACE and a bioaffinity target. In another embodiment, the Alzheimer-disease associated A-beta 1-42 peptide and in particular multimers and complexes of the peptide comprise a bioaffinity target. In another embodiment, any component of an arteriosclerotic plaque is a bioaffinity target.

### Nanoparticles

The particles for this application preferably are paramagnetic or superparamagnetic so as to prevent aggregation in the absence of an external magnetic field. Typically such particles comprise a ferrofluid embedded in a suitable carrier matrix. The individual magnetite particles are typically no larger than 20 nm in size and do not exhibit ferromagnetic behavior even when combined into the matrix of a larger size particle. Superparamagnetic or paramagnetic particles embedded into a suitable matrix will be referred to herein as nanoparticles.

For the DACE capture application, such particles preferentially will comprise a matrix that is compatible with in vivo use, i.e. non-allergenic/non-immunogenic, biocompatible and biodegradable. The nanoparticles also disperse well in the biological fluid into which they are introduced and only aggregate in the presence of an applied magnetic field.

NPs for use in a circulatory system or in the lymphatic system will generally have an average diameter of less than 200 nm, and sometimes also nanoparticles of up to 1 μm size will be used. Preferred particle sizes are larger than 5 nm and less than 1 μm when used in certain biofluids such as blood. There are typically ˜10⁹ nanoparticles per μl of functionalized particle preparation, as in similar, commercially supplied preparations. The nanoparticle preparations may be diluted up to 10⁴ times in injection buffer.

In another preferred embodiment, the preferred particle sizes of nanoparticles that are used for biofluids of body cavities are between 100 nm and 30 μm in size to enhance capture efficiency.

In one embodiment the nanoparticles are paramagnetic. Paramagnetic nanoparticles become magnetized in the presence of a magnetic field and demagnetize slowly when the magnetic field is withdrawn. Thus, these particles do not aggregate until a magnetic field is applied, they disperse once the magnetic field is withdrawn, and they have little tendency to aggregate outside a magnetic field, which is important to avoid generation of larger clots.

In a preferred embodiment the nanoparticles are superparamagnetic. Superparamagnetic nanoparticles become magnetized in the presence of a magnetic field and remain demagnetized when the magnetic field is withdrawn. Thus the particles do not aggregate until a magnetic field is applied. Superparamagnetic nanoparticles are particularly suited for use in the systems and methods described herein since they preserve the surface to volume ratio advantage when the particles disperse and they are not prone to aggregation after being brought in contact with a magnetic field. Aggregation without the presence of an external field could lead to adverse physiological effects, such as embolism.

The shape of the nanoparticles is selected to optimize the biological compatibility of the fNP. Based on general ease of production and availability, in one preferred embodiment, the nanoparticles are in the shape of spheres. In another preferred embodiment, the nanoparticles are elongated. In animal experiments, certain non-spherical particles have been shown to exhibit longer lifetime in circulation in vivo.

The particles may be spherical or non-spherical. In one preferred embodiment, the particles are spherical. In other embodiments, the particles may be non-spherical. For example, the nanoparticles may be oblong or elongated, nanotubes, nanorods, or have other shapes such as those disclosed in U.S. Publication No. 2008/0112886 and WO 2008/031035, entitled “Engineering Shape of Polymeric Micro- and Nanoparticles,” by S. Mitragotri, et al. and/or U.S. Publication No. 2006/0201390, entitled “Multi-phasic Nanoparticles,” by J. Lahann, et al. The average diameter of a non-spherical particle is the diameter of a perfect sphere having the same volume as the non-spherical particle. If the particle is non-spherical, the particle may have a shape of, for instance, an ellipsoid, a cube, a fiber, a tube, a rod, or an irregular shape. In some cases, the particles may be hollow or porous.

Other shapes are also possible, including regular and irregular shapes. Irregular and elongated shapes are less prone to immunogenic recognition and can thereby help increase the lifetime of the fNPs, As such elongated and non-spherical shapes are preferred embodiments. There are indications in the art that highly prolonged particles (e.g.: needles, very long nanorods, carbon-fiber tubes) may elicit toxic responses and are preferably avoided. For the purpose of choosing a suitable size when using non-spherical nanoparticles, the largest length will be of consideration.

Chemical activation of nanoparticles is well known and is used to enable further coating, and chemical modification, including attachment of bioaffinity molecules and other chemical and biological functionalization (Functionalization of nanoparticles). Binding of components can occur through different means, for instance covalently or through non-covalent molecular interactions. The nanoparticles are typically prepared and commercialized to have chemical structures that allow the attachment of organic or anorganic chemical entities. These chemical structures include but are not limited to aliphatic amines, carboxylic acids, alcohol groups, sulfhydryl-groups, biotin, streptavidin, carbohydrates, peptides, polynucleic acid, functionalized polymers such as bi-functionalized polyethylenglycol, or other functional groups designed to further functionalize the particle with additional agents. Such activated variations of nanoparticles as well as custom modifications, and kits to functionalized nanoparticles are known in the art and are often commercially available. Such functionalization enables the attachment of additional functionalization. The necessary procedures and methods to attach primary and secondary functionalization are known in the art. We will consider such pre-functionalization with chemical structures designed to allow further functionalization to be part of the nanoparticle because commercial nanoparticles are often supplied with such activations.

As use herein, a nanoparticle with or without activations but without further coating shall be referred to as a nanoparticle. Nanoparticles with a chemical functionality so as to facilitate further functionalization or coating shall be referred to as “activated nanoparticles”. The generic word particle shall mean any small object with a size less than 30 μm along its shortest axis. It may refer to a particle of any form (such as activated, coated, functionalized, or not, or any combination thereof).

### Biocompatible Coatings

NPs may be coated with one or more biocompatible materials so as not to elicit an immunological response. Coating of nanoparticles are described in the literature and suitable coatings include for example, inorganic layers, organic layers, proteins with or without modifications, sugar moieties, lipids and other biological or non-biological components. Such nanoparticles are commercially available. These particles can be tested for their in vivo use. Typically, a pharmacokinetic study is executed that determines the half-life of such particles in the circulatory system and histopathology is typically used to evaluate the fate of such particles, such as accumulation and/or degradation in liver and spleen.

These coatings are also intended to reduce non-specific binding of the nanoparticles to non-targeted cells or other components of the body fluid and of the circulatory system, such as cell walls and to reduce undesirable aggregation of the particles. Examples of components typically used for surface passivation are, without limitation, polystyrene, proteins, e.g. serum albumin, human serum albumin, bovine serum albumin, modified polyethylene, polyethylenglycol, polyaminoacids, inorganic coatings such as silanes, metals, such as gold, or combinations thereof, which may optionally be chemically linked (often referred to as “crosslinked”). Coatings may be dextran, gold, polymers, sugars, proteins (e.g. albumin, transferring), in particular proteins naturally found in blood, cross-linked proteins such as cross-linked serum albumin, polysorbates, biological membranes, polygalacturonic acid, polyaminoacids. Conventional nanoparticle coating methods⁴⁹ include dry methods such as physical vapor deposition, plasma treatment, chemical vapor deposition, and pyrolysis of polymeric or non-polymeric organic materials for in situ precipitation of nanoparticles within a matrix. Wet methods for coating nanoparticles include sol-gel processes and emulsification and solvent evaporation techniques. Reducing non-specific binding can also be overcome by exposing the surface of the functionalized particles to a solution containing components that saturate unspecific binding sites on the surface but do not interfere with the function of the specific binding moieties.

The nanoparticles can be coated with a polysaccharide polymer or monosaccharide to increase their biocompatibility. This technique provides the advantage of diminishing an immune response to the particles since glycans do not typically illicit such a response (Lacava, et al., Journal of Magnetism and Magnetic Materials, 272-276, 2434-2435 (2004)). The polymer coating also contains numerous free hydroxyls that willingly form hydrogen bonds in aqueous solution. In concert, the many surface hydroxyls hold the particle and surface coat in suspension for an indefinite period of time. The coating is preferred in those embodiments in which the nanoparticles are injected into the general circulation or the ascites fluid of the peritoneal cavity.

Suitable coating materials include, but not are not limited to, silanes, such as polydimethylsiloxane, silicon oil, silicones, vinylsilane graft copolymers, in which a biocompatible material is grafted to the vinyl silane, such as those listed above; saccharides, polysaccharides, and derivatives thereof, such as dextran, glucuronic acid, polygalacturonic acid, chitosan, neuraminic acid, agar, agarose, alginates, carrageenan, celluloses and modified celluloses, condroitin, hyaluronic acid, pectin, starch, xanthan, and combination thereof. Alternative coating materials include, but are not limited, to non-degradable, biocompatible polymers, such as poly(alkylene oxides), such as PEG, PPO, and copolymers thereof, polyurethanes, biocompatible acrylates and alkylacrylates, such as methacrylates and hydroyalkyl methacrylates, polyalkylenes, such as polyethylene, polypropylene, and polytetrafluoroethylene, polyvinyl alcohols, polyvinylacetates, poly(ethylene-co-vinylacetate), polyesters, such as poly(ethylene terephthalate), poly(sulfones). Alternative coating materials include, but are not limited, biodegradable, biocompatible polymers, such as PLA, PGA, and copolymers thereof, poly(p-dioxanone) and copolymers thereof, polycaprolactone, polyhydroxyalkanoates, polyanhydrides, poly(orthoesters), polyphosphazines, poly(alkylcyanoacrylates), and proteins, such as gelatin. Further, the coating may contain Surfactants, such as Tweens, poloxamers, pluronics, etc.

In other embodiments the nanoparticles comprise a polymeric matrix. In one embodiment, the polymeric matrix comprises two or more polymers. In another embodiment, the polymeric matrix comprises polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In still another embodiment, the polymeric matrix comprises one or more polyesters, polyanhydrides, polyethers, polyurethanes, polymethacrylates, polyacrylates or polycyanoacrylates. In another embodiment, at least one polymer is a polyalkylene glycol. In still another embodiment, the polyalkylene glycol is polyethylene glycol. In yet another embodiment, at least one polymer is a polyester. In another embodiment, the polyester is selected from the group consisting of PLGA, PLA, PGA, and polycaprolactones. In still another embodiment, the polyester is PLGA or PLA. In yet another embodiment, the polymeric matrix comprises a copolymer of two or more polymers. In another embodiment, the copolymer is a copolymer of a polyalkylene glycol and a polyester. In still another embodiment, the copolymer is a copolymer of PLGA or PLA and PEG. In yet another embodiment, the polymeric matrix comprises PLGA or PLA and a copolymer of PLGA or PLA and PEG. U.S. Pat. No. 8,273,363 discloses methods of producing nanoparticles with such coatings.

In another embodiment, the polymeric matrix comprises a lipid-terminated polyalkylene glycol and a polyester. In another embodiment the polymeric matrix comprises lipid-terminated PEG and PLGA. In one embodiment, the lipid is of the Formula V. In a particular embodiment, the lipid is 1,2 distearoyl-sn-glycero-3-phosphoethanolamine (DSPE), and salts thereof, e.g., the sodium salt.

In an embodiment of the methods described above, the copolymer is a copolymer of PLGA and PEG, or PLA and PEG. In another embodiment, the first polymer is a copolymer of PLGA and PEG, wherein the PEG has a carboxyl group at the free terminus. In another embodiment, the first polymer is first reacted with a lipid, to form a polymer/lipid conjugate, which is then mixed with the low-molecular weight PSMA ligand. In still another embodiment, the lipid is 1,2 distearoyl-sn-glycero-3-phosphoethanolamine (DSPE), and salts thereof, e.g., the sodium salt.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEGylated polymers and copolymers of lactide and glycolide (e.g., PEGylated PLA, PEGylated PGA, PEGylated PLGA, and derivatives thereof. In some embodiments, polyesters include, for example, polyanhydrides, poly(ortho ester) PEGylated poly(ortho ester), poly(caprolactone), PEGylated poly(caprolactone), polylysine, PEGylated polylysine, poly(ethylene inline), PEGylated poly(ethylene imine), poly(L-lactide-co-L-lysine), poly(serine ester), poly(4-hydroxy-L-proline ester), poly[a-(4-aminobutyl)-L-glycolic acid], and derivatives thereof.

Those of ordinary skill in the art will know of methods and techniques for attaching the coatings covalently to the Activated nanoparticle, for example, by using EDC (1-ethyl-3-(3-dimethylaminopropyl)carbodiimide hydrochloride) and NHS (N-hydroxysuccinimide) to react a polymer to an amine, by ring opening polymerization techniques (ROMP), or the like.

In a preferred embodiment, the nanoparticle is coated with PEG molecules that are at least 4 monomeric units long and less than 100 monomeric units long. Preferably the PEG molecules are less than 24 monomeric units long, and more preferably the PEG units are about 12 monomeric units long. Preferably the PEG units are longer than 6 monomeric units. Furthermore the preferred PEG molecules are bifunctionalized as to facilitate their chemical coupling to the encapsulated or non-encapsulated nanoparticle core on one end and a further functionalization with other molecules, including linking of the bioaffinity molecule or a florescence marker or any other functionalization suitable for a particular purpose.

### Functionalization of the NPs

At a minimum, the nanoparticles will be functionalized with at least one component that can recognize a particular DACEs that is to be targeted. A used herein an fNP will be any nanoparticle that comprises at least one such bioaffinity molecule.

The functionalization is accomplished through a suitable linker chemistry or non-covalent adhesion. In a preferred embodiment, the linkage will be a covalent bond. In a particular preferred embodiment, the linkage will be trough a bi-functional linker. Suitable linkers and chemical reactions are known in the art and are directly dependent on the choice of the nanoparticles and the bioaffinity molecules. For linking of any antibody or other protein to nanoparticles, suitable linkers and chemical reactions, as well as analytical techniques to monitor the success of such reactions have been described and compared⁶⁹ and reagents are offered by as commercial kits.

The density of the bioaffinity molecule is preferably such that a large portion of the surface of the nanoparticle is covered. It is known in the art that the density can be adjusted by the relative concentration of the reactants used in the linking reaction, and parameters of the linking chemistry such a temperature and time of the reaction. To optimize the density, in vitro binding of the fNPs to a DACE can be measured. In a preferred embodiment, there is at least one bioaffinity molecules per 1000 nm̂2 of nanoparticle surface area. In a preferred embodiment, an fNP of approximately 50 nm in diameter shall have about 3-10 bioaffinity molecules.

Methods to Establish a Complex Between the fNPs and the DACE

The interaction between the fNPs and the DACE will be mediates through complex formation between the bioaffinity molecule that comprise the fNPs and the bioaffinity target that are present on the DACE.

The functionalization and coating can also be such that the nanoparticles will be preferably be taken up by the DACE, for example via endocytosis, including receptor-mediated endocytosis.

The binding or uptake of the fNP to the DACE may induce cellular changes that can increase immunogenicity or apoptosis, or other cellular processes that can lead to elimination or weakening or death of the DACE. Such secondary biological effects are tolerated and desired as they will also lead to the desired reduction in viable DACEs in the body.

In one embodiment, the nanoparticles are designed to have one or more interactions of an antibody or bioaffinity molecule on its surface to interact with complementary regions on the DACE. These may involve the same antigen-antibody interaction or may involve more than one different epitope and their complementary antibodies. Such antibodies may also be bifunctional antibodies.

In another embodiment, the bioaffinity interaction between the fNP and the DACE may be mediated or may include a ligand-receptor interaction, for example, the interaction of a growth-factor receptor and a molecule binding to the growth factor receptor, e.g. a complementary ligand, protein, or drug.

For example, in one embodiment the structure is a receptor-tyrosine kinase-binding molecule that can direct the fNP to a DACE that has high levels of receptor-tyrosine kinase expression (e.g. a HER2 positive tumor cell.)

The functionalization of a nanoparticle may include components in addition to the bioaffinity functionalization (We refer to such additional components as secondary functionalization.). The secondary functionalization may include components that allow to detect the particle, identify the particle, render the particle suitable for endocytosis by the cells, make the particle detectable by a device such as a magnetic resonance imaging device or a computer tomography device or an ultrasound device, or make the particle therapeutically active by functionalization with a drug-like molecule that damages the DACE.

In certain embodiments, the secondary functionalization is a prodrug that can be activated by light, cellular metabolism, including e.g., change in pH upon endocytosis or uptake into lysosomes, or other physical-chemical or chemical process. In yet another embodiment, the secondary functionalization may include a molecule that renders the cell sensitive to cell death by use of a physical process such as a photodynamic process, an electromagnetic heating process, or similar that utilizes the secondary functionalization together with a physical, or physical-chemical process to create damage to the target cells that will result in cell death, or phagocytosis or similar degradation of the DACEs.

In a preferred embodiment a base functionalization is streptavidin. In this case, additional functionalization can easily be achieved by adding biotin-labeled molecules that introduce additional specific functionalization of the nanoparticles.

Functionalization can be attached directly to the nanoparticles, or attached via “spacers”, organic molecules such as polyethylenglycol, polyamino-acids, or secondary antibodies, designed to bind another functionalization.

The interaction between an fNP and a DACE can be through a single molecular interaction, or can include multiple interaction sites. It is desired to achieve quasi-irreversible binding. The equilibrium constant (k) as defined in physical chemistry (delta G=R*T*ln(k)) between the DACE and the nanoparticles shall be less than 10exp(−9)/mol. If multiple interactions are created for an fNP-DACE pair, the binding affinity will be strongly enhanced. In a first approximation, the binding constant of individual interactions will multiply to result in the overall nanoparticle-DACE binding affinity.

Such secondary functionalization may be attached to the surface of the particle or embedded in the particles and may be one or more of metals, metal ions, dyes and fluorescent molecules, components detectable by magnetic fields such as used in MRI, components detectable by tomography (CAT), components detectable by in vivo or in vitro luminescence, or other components that allow analytical detection or imaging.

The fNPs bound by a DACE may reside on the surface of the DACE or may be taken up by the DACE, e.g. by endocytosis, receptor internalization or a similar natural biological process.

Preferred embodiments include multiple functionalizations such as multiple copies of the same functional group or a mix of multiple different functionalizations such as an antibody and an imaging function, or a DACE-binding component and a cytotoxic component.

In another embodiment, the functionalization increases the probability for phagocytosis of the fNP-labeled DACEs by the immune system.

In another embodiment, the fNPs cause the DACE to undergo apoptosis.

The fNPs may optionally be modified to enhance its lifetime in the organism using functionalization such as proteins (e.g. serum albumin) or inorganic coatings, such a gold coating.

### Complex Formation

An fNP, by design, will complex with a targeted DACE moderated by the interaction between the bioaffinity molecule and the bioaffinity target to form an fNP-DACE complex. Such intermolecular complex formations are dominated by two basic physicochemical principles: the kinetic and the thermodynamic of the complex formation.

The thermodynamic aspect is that of the binding affinity of the complex which is guiding the selection of the bioaffinity target and bioaffinity molecule part (see above). By design, the binding affinity is very strong. As such the kinetic off-rate is typically very slow or negligible in particular since the binding partners have been selected to have a very strong binding affinity and since multiple pairwise interactions within each fNP-DACE complex are typically found.

The kinetic of the complex formation, i.e., the on-rate, however, is an important consideration in the design of the system. In a typical molecular system, biologically relevant molecules such as metabolites and proteins are diffusing and rotating very rapidly within a biofluid and the relative motion is caused by the Brownian motion of both binding partners. Thus, for a typical biological system e.g. a metabolite present at a few micromole/1 will “find” a protein in milliseconds or less (“diffusion controlled processes”). However, the Brownian motion is not effective for larger biological systems such as cells. Similarly nanoparticles have a reduced rate of fluctuation when compared to a soluble protein, for example. While smaller nanoparticles such as a Nanoparticle of less than 100 nm in size will still have a significant amount of Brownian motion, larger Nanoparticles, e.g. those of approximately 1000 nm or more in size, will only very slowly find an interaction partner of similar size sole bases on Brownian motion. Active mixing of a fluid (stirring or even flow through a circulatory system) will also move those particles in a largely parallel manner. As such, diagnostic kits where such larger nanoparticles are used to complex with cells require hour or day-long equilibration die to the reduced mobility of the two binding partners.

In a preferred embodiment, more than one fNP will bind to the surface of a DACE.

Such complexation may occur through the adhesion of the fNPs to the DACEs, or through uptake of nanoparticles by the DACE via cellular metabolism, membrane transport, endocytosis or similar, naturally occurring cellular processes. The objective is to bind or accumulate one or more nanoparticles to the DACE. In a preferred embodiment multiple nanoparticles complex with the DACE in order to enhance the magnetic force between the magnetic capture device and the fNP-DACE complex.

Based on models of steric hindrance, and limited by the density of the bioaffinity targets on the DACE, and the relative size of the DACE and the fNP, there may be one fNP or multiple fNPs binding to the surface of a DACE. For example, assuming an approximate diameter of 10 μm for a typical circulating cell, and further assuming an approximate diameter of 50 nm for a small fNP, about 10000 fNPs may bind until a complete coverage of the DACE has been achieved. As such, it is possible to achieve very high loading of fNPs on the surface of a DACE which can greatly enhance the ability to concentrate and capture the resulting complexes of fNPs and DACE.

DACEs that are considerably smaller that a cellular or other larger biological system such as proteins and other low-to high molecular weight molecules are not likely to bind more than one or a few fNPs. In those cases, the use of larger fNPs such as those of hundred nanometers or several hundred nanometers to a few thousand nanometers are preferred. Those fNPs can bind many DACE and are more easily captured by a magnetic device.

In an alternative embodiment, the fNPs are designed to be incorporated into a target cell. DACE, in particular those with a highly active metabolism such as tumor cells and bacterial cells and cells specifically designed to take up entities bound to their surface such as NK cells are particular prone to uptake of entities that bind to their surface. As such, it is a preferred embodiment to generate a high enrichment of fNPs to accumulate within a DACE. It has been shown in the literature that cellular uptake is efficient when fNPs bind to a surface epitope of a cell. For example, endocytosis is a biological process that achieves such uptake of entities bound to the surface of a cell.

DACE that have been loaded (by surface adhesion or uptake) with more than one fNP are preferred as they will be more likely to be captured at a location of a high magnetic field. As such, it is preferred to have more than one, and preferably more than 5 and more preferably more than 10 fNPs in complex with a DACE that is of large enough size to allow for such additional interactions.

In another embodiment, improved binding of the targeted cells to the fNPs is achieved through the use of multiple bioaffinity molecules (of the same or different type) that simultaneously bind the targeted DACE to the fNPs (i.e. two or more bioaffinity molecules on one nanoparticle will bind to two or more bioaffinity target of an individual DACE.) Such cooperative binding allows for both a significantly higher on-rate of binding (improved kinetics) as well as an improved binding affinity.

In a preferred embodiment, multiple fNPs bind simultaneously to a DACE to increase the efficiencies of concentration, capture and immobilization of complexes of fNPs and DACE.

In another embodiment, the fNPs consist of a cluster of individual fNPs. Such a group has increased surface and motional flexibility to allow for additional interactions. Furthermore, members of the nanoparticle cluster can contribute different functionalization that can help to enhance binding affinity and binding specificity. In the latter case, specificity is generated by allowing that different, weakly binding functionalization (with k between 1 millimolar and 1 nanomolar) contribute together to a strong binding affinity (k less than 1 nanomolar). As such, DACEs that are characterized by more than one epitope can be selected. For example, currently, many cancer stem cells are characterized by multiple epitopes.

In yet another embodiment, the nanoparticles form chains, pairs, or small clusters of 2-1000 nanoparticles, preferably with an average number between 1-100 nanoparticles per cluster, and most preferably with an average number between 5-20 nanoparticles per cluster. Those clusters can be generated and their size (i.e. the number of individual nanoparticles forming a cluster) can be controlled by the choice and relative concentration of linking. Linking modules are functionalizations added to the nanoparticles that create a defined bond between two nanoparticles. In one embodiment, single-stranded DNA molecules are added to one part of a nanoparticle population, a complementary strand of DNA is added to another nanoparticle population. Mixing of these two populations will create aggregates between particles of both populations. Other complementary pairs of linking Molecules can be selected from other complementary pairs of molecules that form specific pairs of linkages. The choice of such linking molecules will also be guided by considerations such as the need to avoid interference with other functionalization on the nanoparticles. Care has to be taken to either saturate or block free binding partners before in vivo use or chose linking partners that are not present in the biofluid or the body part within the particles may be used. Suitable pairs of binding partners are well known in the art. They may be chosen from pairs of antibodies/antigen, streptavidin/biotin, receptor/ligand pairs, interacting proteins such as lysine-zipper, homo- and heterodimerizing protein pairs, etc. In another preferred embodiment, on linking partner is an antibody against the Fc fragment of the antibody used as a bioaffinity molecule. For example if the bioaffinity molecule is an anti-human-EpCAM antibody created in a mouse, it has a mouse FC fragment. An antibody against the mouse FC fragment on another antibody population can bind one or more such anti-human EpCAM fNPs to generate clusters of nanoparticles. The stoichiometry of the reactants on each nanoparticle population and the ratio of the population will, according to statistical probabilities. The two populations use I the mixing may each contain the exact same population of nanoparticles or any mix of nanoparticles and functionalization of such nanoparticles. For the purpose of choosing a suitable size when using non-spherical nanoparticles, the smallest and the largest length will be of consideration. Single-bodied nanoparticles are preferably smaller than 30 μm on their longest extension. Nanoparticle clusters that are created by flexibly linking of multiple individual nanoparticles are to be measured along their shortest axis if they are being used in a system where passage through small capillaries is a requirement, such as being injected into the circulatory system. For example, a minimally branched chain of nanoparticles shall be treated for sizing purposes as if the size is that of an individual nanoparticle with the underlying assumption that.

Introduction of the Beads into the Body

The fNPs can be introduced in a variety of manners, as known in the medicinal art most notably intraperitoneally (to bind to residual malignant cells following abdominal surgery for cancer), or intravenously (to bind to blood-borne DACE) or by injection into other suitable body fluids or body cavities.

Preferably, the superparamagnetic nanoparticles are administered in a sterile suspension, in a suitable carrier (referred to also as formulation). The carrier is a fluid which is physiologically compatible with the subject undergoing treatment.

In one preferred embodiment, the vehicle comprises an isotonic phosphate-buffered saline (PBS) solution for injection into the circulation. Optionally, the carrier also contains heparin to prevent coagulation of the blood in the system. In another embodiment, the carrier also contains an effective amount of an antibiotic, such as penicillin or ampicillin, to reduce any bacterial growth which may be associated with the nanoparticles. The carrier is also preferably formulated such that it is at physiological pH. In some instances, in particular with larger nanoparticles, it may be necessary to agitate the nanoparticle suspension to ensure that the nanoparticles are relatively uniformly dispersed in the carrier.

To initiate a treatment, the subject (e.g. patient) will be given, e.g. by intravenous injection, a small volume of carrier fluid (typically phosphate buffered saline solution (PBS), or injection buffer) containing fNPs of up to 1% solids, more generally about 0.1-1% solids, and preferably diluted to about 0.01-0.1% solids. An excess of fNPs is injected depending on the disease load with target DACEs. Preferably an amount of fNPs is injected that corresponds to at least 1000 times, and preferably at least 10.000 times, and more preferably at least 10 exp 5 times as many fNPs as target DACEs are expected to be present. For example, there are typically between 1000 and 10 million CTCs in a patient with cancer (equivalent to one to a few thousand CTCs per milliliter of blood).

Preferably, particle sizes and doses are selected that achieve a nanoparticle density in the body fluid that is characterized by a mean distance between adjacent nanoparticles of less than 100 times of the size of the nanoparticle, or less than ten times the size of a CTC. Injecting 100 μl of a 1% suspension of 500 nm nanoparticles provides about 10 exp 9 particles per liter of blood in an adult, resulting in a mean distance of less than 100 μm between the particles. Injecting 100 μl of a 0.1% suspension of 50 nm nanoparticles provides about 10 exp 11 particles per liter of blood in an adult, resulting in a mean distance between the particles of approximately the size of a CTC.

### Removal

The particles are captured using a magnetic field gradient that magnetizes, captures and holds the fNPs and any associated DACEs inside a defined volume.

In one preferred embodiment for DACE removal from circulation, the arrest is achieved by a strong magnet applied to the skin above a vein. This will also trap the DACE that are now bound to the nanoparticles. The blood flow is then stopped for that section of the vein, the magnet is removed to allow suspension of the captured particles in that volume, followed by a blood draw (venipuncture) of that volume to remove the suspended DACE.

### Therapeutic System, Objective and Utility

It is the objective of the present invention to enable treatment of DACE-associated diseases. The therapeutic system can be adjusted by the choice of the bioaffinity molecules to target specific DCAEs and thus treat a given disease.

The therapeutic system comprises
a. A population fNP selected to target a specific DACE through one or more specific bioaffinity molecules
b. Introducing a formulation of the fNPs into a biofluid of a subject
c. Allowing for or, optionally, actively enhancing the complexation of fNPs and DACEs through the use of magnetic mixing

d. Concentration and capture of the fNP-DACE complexes by a magnetic capture device

e. Removal of the captured complexes from the subject.

The procedure to test and establish a particular therapeutic implementation comprises in vitro optimization of the components, preclinical safety and efficiency studies of the fNPs and the therapeutic system in model organisms, and clinical trials of the therapeutic system.

In a preferred embodiment, the therapeutic system may be used as an adjuvant therapy to complement or supplement other treatment options. For example, in treatment of a cancer patient, the local treatment of a tumor (e.g. by surgical removal or ablative methods) is preferably followed by a CTC count to establish the need of a CTC removal. Isolation of CTCs for diagnostic purposes to establish suitable bioaffinity molecules through molecular analysis is part of a preferred embodiment. In other examples, the therapeutic system of this invention will be complemented with systemic treatments such as treatments with anti-viral or anti-bacterial or lipid-lowering or other medication for a given disease diagnosis.

In a preferred embodiment the therapeutic system will be supplemented with a diagnostic method to detect the presence of DACE before the use of therapeutic system. In a preferred embodiment the application of the therapeutic system in a patient is followed by an analysis of the success of removing of the DACEs in the treated biofluid. In a further embodiment the therapeutic system comprises the molecular analysis of the captured and removed DACE. In a further preferred embodiment the therapeutic treatment is followed by regular diagnostic analyses to monitor possible recurrence of the DACE in the patient. Repeat treatment after recurrence or partial removal comprises the therapeutic system. Concurrent and complementary treatment with other local or systemic therapies also comprises a preferred therapeutic system.

In a cancer patient, the therapeutic system will be used if CTCs are detected or it can be used prophylactically even without CTC detection. In both cases, regular CTC count surveillance of the patient may be performed to monitor immediate success of the CTC removal and to monitor a possible recurrence or outbreak of CTCs. Detection of CTCs may indicate the need for a repeat application of the therapeutic system described in this invention or alternative or complementary treatment.

For the purpose of a clinical trial and, DACE counts in a patient and molecular characterization of the DACE comprise a biomarker to monitor the efficacy of the therapeutic system and as such a biomarker analysis becomes part of the therapeutic system. Such a biomarker arm of a clinical trial enables a timely monitoring of clinical success which represents critical secondary endpoint that enables efficient implementation of the necessary clinical studies that are part of the regulatory path such a therapeutic system needs to pass.

In a preferred embodiment of the therapeutic system a CTC will be provided for a patient prior and following the procedure. In a more preferred embodiment, the CTC diagnostic will be performed regularly and repeat application of the therapeutic removal of CTC will utilized.

The advantage of the in vivo capture is the ability to provide a high density of fNPs relative to the CTCs count in circulation and the ability to equilibrate and capture CTCs over several days through techniques such as magnetic mixing.

The present invention is able to diagnose or treat patients for diseases that are caused or associated with components in circulation.

In one embodiment, the bioaffinity target is a member of the CD28/CTLA-4 family if T-cell regulators. These proteins are expressed on T cells, B cells, or macrophages. The complementary ligands, can thereby be selected as bioaffinity molecules. In one embodiment, the bioaffinity target is Programmed cell death protein 1 also known as PD-1 or CD279. The corresponding ligands PD-L1 and PD-L2 comprise two examples of a complementary bioaffinity molecule for PD-1. PD-L1 and PD-12 are also expressed on specific cell lines. For example, PD-L1 is expressed on almost all murine tumor cell lines. PD-L1 thus represents a suitable bioaffinity target that can be recognized with PD-1 or a fragment thereof.

In one preferred embodiment, the Therapeutic System comprises a bioaffinity molecule that is an Antibody-Like molecule against PD-1. In a more preferred embodiment the bioaffinity molecule is a humanized antibody against PD-1. This therapeutic system is directed against non-small-cell lung cancer, melanoma, and renal-cell cancer. For example, in a clinical trial with a Monoclonal antibody against PD-1, BMS-936558, produced complete or partial responses in non-small-cell lung cancer, melanoma, and renal-cell cancer, in a clinical trial with a total of 296 patients. Colon and pancreatic cancer did not have a response⁷⁰.

In another preferred embodiment, an antibody against PD-L1 or the extracellular domain of PD-1 comprises a bioaffinity molecule for fNPs directed against e.g. myeloma.

The murine form of the antibody or PD-1 fragment represents a suitable model for preclinical optimization of the therapeutic system. In one preferred embodiment, an fNP comprising a bioaffinity molecule directed against human PD-L1 is used for the preclinical safety evaluation of the particles. The preclinical species for the study is a mouse expressing human PD-1. Methods to genetically humanize mice are known in the art and such models are commercially available.

Other preferred embodiment uses bioaffinity molecules directed against homologue bioaffinity target in mouse or rat or dog or Guinea pig or rabbit or other preferred preclinical species for preclinical studies.

In another preferred embodiment the preclinical species will be genetically manipulated as to express the human form of the bioaffinity target for preclinical studies. (Those skilled in the art know that replacement of a endogenous gene with a human homologue is currently limited to very few species, such as a few mouse strains, and with limitations in some rat strains).

In a preferred embodiment, the nanoparticle core is coated with a PEG linker. In a particular embodiment, the nanoparticle comprises superparamagnetic nanoparticles (e.g. SPIONs), encapsulated in cross-linked Dextran and further functionalized with bifunctional Peg-12 and further functionalized with an antibody and, optionally further functionalized with one or more of a fluorescent marker, a PET-active marker, a drug molecule, a radioactive marker. Such nanoparticles are available from commercial vendors and methods for production of the core particle, their encapsulation and the linker chemistry is well known in the art. Useful diagnostic radioisotopes exist, and are well-known to those ordinarily skilled in the art. The useful diagnostic and therapeutic radioisotopes may be used alone or in combination.

While the foregoing written description of the invention enables one of ordinary skill to make and use the invention, those of ordinary skill will understand and appreciate the existence of variations, combinations, and equivalents of the specific embodiment, method, and examples herein.

The invention should therefore not be limited by the above described embodiment, method, and examples, but by all embodiments and methods within the scope and spirit of the invention. All patents, patent applications and publications referred to in this application are herein incorporated by reference in their entirety.

The following examples set forth the general procedures involved in practicing the present invention. To the extent that specific materials are mentioned, it is merely for purposes of illustration and is not intended to limit the invention.

In one embodiment to help prevent formation of distant metastasis from the primary tumor the technology comprises the use of paramagnetic nanoparticles that are functionalized to bind to at least one cell surface marker, such as the epithelial cell adhesion molecule (EpCAM), which is present on CTCs of the specific cancer that is to be treated (such as breast, lung, prostate, gastrointestinal cancers). These fNPs are introduced into a subject. A weak magnetic or electromagnetic field and mechanical methods may be applied to enhance binding kinetics of fNP-DACE interactions. In a further step of the embodiment, particles with or without attached DACEs are collected by a magnetic field strong enough to collect the fNPs and their associated CTCs in a defined location in the body, e.g. a section of a vein by applying a strong laboratory magnet to the area. Such magnets are known in the art where they are used typically for collecting paramagnetic nanoparticles from laboratory samples.

The invention is designed to reduce the number of circulating DACEs in order to cure a patient or reduce progression of disease. The method provides the ability to mechanically remove disease-causing biological material from circulation to prevent the disease progression. The method is minimally invasive and simple enough to use in geographic regions with limited medical infrastructure. This method supplements existing local (e.g. surgical) and systemic (e.g. radiation and chemotherapy) treatments. The approach to therapeutically remove DACEs from the body bypasses (yet also complements) the need for genomic profiling of DACE-diagnostics and the limitations of current personalized treatment options²². It therefore is more broadly applicable and particularly useful when prognostic and therapeutic options are not or not yet available for patient treatment—in particular during early disease stages where stopping the spread of disease by the removal of DACEs can prevent the establishment of secondary disease phenotypes, such as metastases.

It is also an objective of the therapeutic system to modify the DACEs through forming a complex with a fNP so as to make the DACE less pathogenic than compared to its original, uncomplexed state. It is another objective to render such DACE-nanoparticle complexes more likely to be recognized and successfully removed by the immune system, in particular by the reticulo-endothelial system. It is yet another objective of the therapeutic system to accumulate nanoparticles in target cells or in accumulations of target cells and disease-associated cellular aggregates including—but not limited to—metastases, plaques, atherosclerotic plaques, centers of inflammation, tumors, blood clots, or fibrous tangles. Such accumulation may occur naturally through processes such as direct complexation of the nanoparticle via the bioaffinity molecule, or through the uptake of a DACE-nanoparticle complex through natural processes such as cell metabolism, endocytosis, or infiltration.

In contrast to diagnostic methods, the removal of DACEs from circulation does not require the highly selective recovery of the native and molecularly undisturbed DACEs. There is no need to subset and profile the disease-causing cells. While diagnostic methods are only of therapeutic value if specific treatment options are available, the current invention may not require any further systemic treatment such as drug therapy because the removal of DACEs eliminates the disease-causing mediator from the body.

### EXPERIMENTAL PROCEDURES

### Example 1

### Production of fNPs

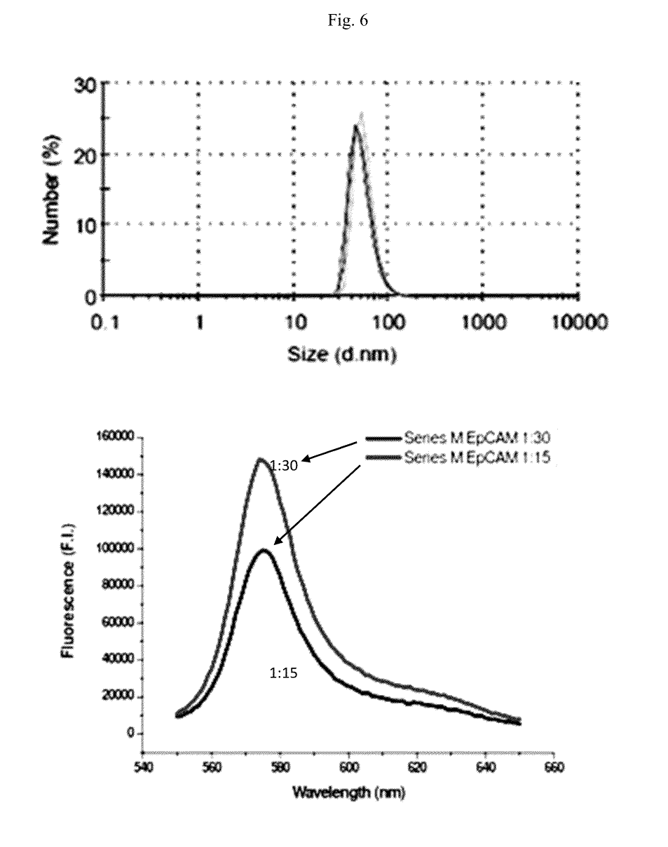

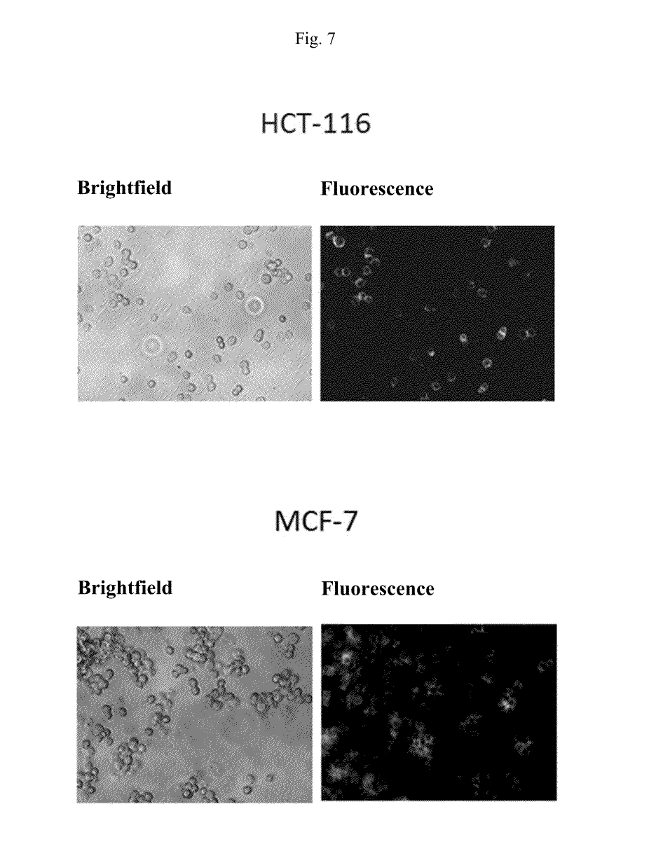

Superparamagnetic ironoxide nanoparticles containing a NH2 activation (CANdot Series M aq, ca, Hamburg, Germany) were obtained. A phycoerythrin (PE)-labeled human-EpCAM antibody (Miltenyi Bioscience, Germany, 130-091-253) was slightly reduced and linked via a SM(PEG)12 linker (Thermo-Fisher Scientific, Rockford, Ill. #22113) to the nanoparticles⁶⁹. The nanoparticles are formulated in PBS suspension at a concentration of 10̂-6M to (about 1 mg/ml Fe) and purified by ultrafiltration and sterile filtration. The resulting particles have a size of 50 nm+−10 nm and show the strong fluorescence spectrum, typical for PE (FIG. 6). The particles are labeled fNP-2.

### Example 2

### Validation of Antibodies and Cell Lines

The following cell lines were used (available from American Type Culture Collection, ATCC, Manassas, Va.):

| | | |
|---|---|---|
| a. | MCF-7 | (human breast carcinoma) |
| b. | HCT-116 | (human colon carcinoma) |
| c. | Caco-2 | (human colon carcinoma) |
| d. | SEM | (leukemia) |

The cells were grown in a Petri plate, the growth media was removed, the cells washed twice with PBS buffer and separated by mild trypsination. About 1 million and 3 million cells per ml were obtained. The cells were centrifuged and suspended in 100 μl PBS buffer. A test sample (50 μl) of).

10 μl of human CD326(EpCAM)-Ab, FITC labeled, (Miltenyi order Nr. 130-096-415) was added to 100 μl of suspended cells. The results (Table 1) demonstrate that the chosen antibody produces strong staining and that the human tumor cell lines are all EpCAM positive. For the purpose of the in vitro validation, cells with low cell aggregation were chosen to facilitate analysis by flow cytometry/FACS.

TABLE 1
Results from light and fluorescence microscopic evaluation of cell lines
stained with h-EpCAM-FITC antibodies.

| | | | Fluorescence |
|---|---|---|---|
| | | | by EpCAM- |
| Cell line | Origin | Aggregation | FITC |
| MCF-7 | human breast | partial | strong |
| | carcinoma | aggregation | |
| HCT 116 | human colon | Few cell | strong |
| | carcinoma | clusters | |
| Caco-2 | human colon | prominent | very strong |
| | carcinoma | aggregation | |
| SEM | mouse | none | none |
| | leukemia | | |

### Example 3

### Validation of fNP-DACE Complex Formation

Cell lines HCT-116 (colon carcinoma cells), BXPC-3 (pancreas carcinoma cells), SU8686 and PANC1 (available from American Type Culture Collection, ATCC, Manassas, Va.) were grown using standard procedures and prepared as in Example 2. The cells were incubated in an eight-well chamber slide in 100 μl medium with 1 μl fNP-1 (h-EpCAM) MicroBeads, Miltenyi Bioscience #130-061-101) or 2 μl fNP-2 (See Example 1) for about 10-20 min at room temperature. Thereafter the cell nuclei were stained with DAPI (blue) for 5 min.

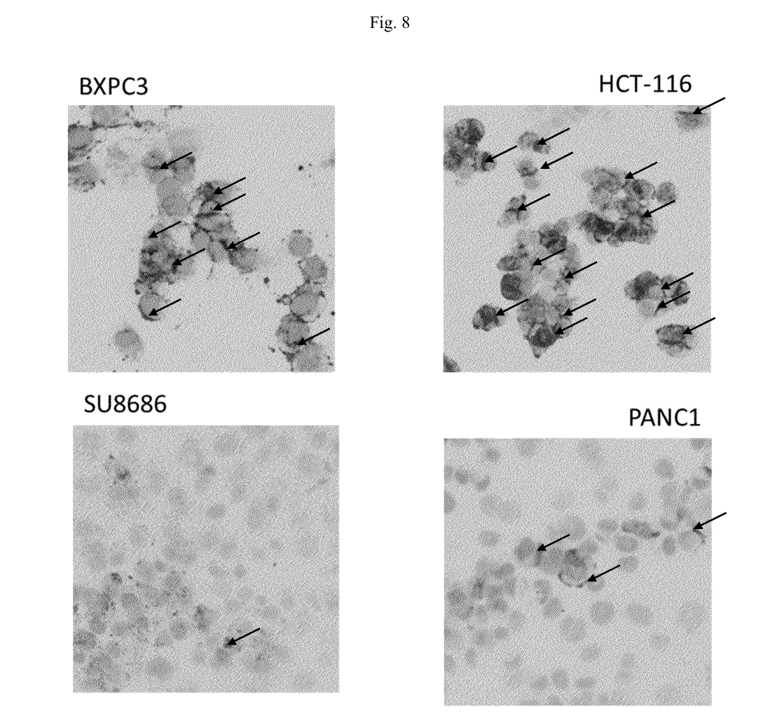

Under fluorescence microscopy, HCT-116 (colon carcinoma cells) show strong staining with fNP-1 and fNP-2 (FIG. 8). BXPC3 (pancreas carcinoma cells) show strong staining with fNP-1 and fNP-2 as well as possible internalization of the fNPs (FIG. 8). SU8686 and PANC1 (both pancreas carcinoma cell lines) are only weakly stained with both fNP-1 and fNP-2 (FIG. 8). SEM cells (acute lymphoblastic leukemia) were introduced as negative controls (no h-EpCAM expression) and show no staining

### Example 4

### Quantitative Comparison of fNP-DACE Complex Formation

BXPC3 cells were prepared as in Example 2 and suspended in PBS/1% FKS to a concentration of 100,000 cells/ml. Decreasing concentrations of fNP-1 (see Example 3) and fNP-2 (see Example 1) were prepared by serially diluting the fNPs up to 10̂7-fold. 1 μl of these were then added to 100 μl aliquots (10,000 cells) each of the BXCP3 stock and incubated over ice for 30 min. 200 μl PBS was added and a FACS analysis was run to collect exactly 4,000 events for each dilution.

Table 2 summarizes the results. Labeling of the BXCP3 cells, as determined from the corresponding scatter plots (not shown), was uniform at all dilution factors of fNPs, i.e. there were no distinct populations of labeled and unlabeled cells present in a single sample.

TABLE 2
Median phycoerythrin signal for BXCP3 cells after 4,000 events in a
FACS/flow cytometer obtained after incubation with increasing dilution
factors of fNP1 and fNP2, respectively.

| | | median PE | |
|---|---|---|---|
| | | (phycoerythrin) signal | |
| fNP-dilution factor | | fNP-1 | fNP-2 |
| 10\hat{ } 0 | 1 | 20020 | 37128 |
| 10\hat{ }-1 | 0.1 | 2452 | 4182 |
| 10\hat{ }-2 | 0.01 | 215 | 566 |
| 10\hat{ }-3 | 0.001 | 61 | 105 |
| 10\hat{ }-4 | 0.0001 | 40 | 46 |
| 10\hat{ }-5 | 0.00001 | 38 | 38 |
| 10\hat{ }-6 | 0.0000001 | 38 | 37 |
| 10\hat{ }-7 | 0.000000001 | 40 | 37 |
| neg. control | 0 | 37 | 37 |

### Example 5

### Characterization of Cells Recovery from Whole Blood

A suspension of HCT-116 cells (Human Colon Carcinoma cells), prepared as described in Example 2 was spiked at 1000 cells/ml, 10,000 cells/ml, and 100,000 cells/ml) into aliquots of whole blood stabilized by citrate. 10 μl of a 1% suspension of fNP-1 in PBS was added and the cells were magnetically captured and washed once with 1 ml PBS buffer and released from the magnet. No further processing was performed with the retained cells and fNP-cell complexes. The retained samples were stained with a) EpCAM-PE for CTC cells, b) CD45-APC for hematopoietic cells, and c) PI for dead cells. The cell numbers used to spike in (1,000-100,000) was chosen to be on the order of typical loads expected for CTC counts in patients, which are several orders of magnitude lower than those of endogenous cells in whole blood.

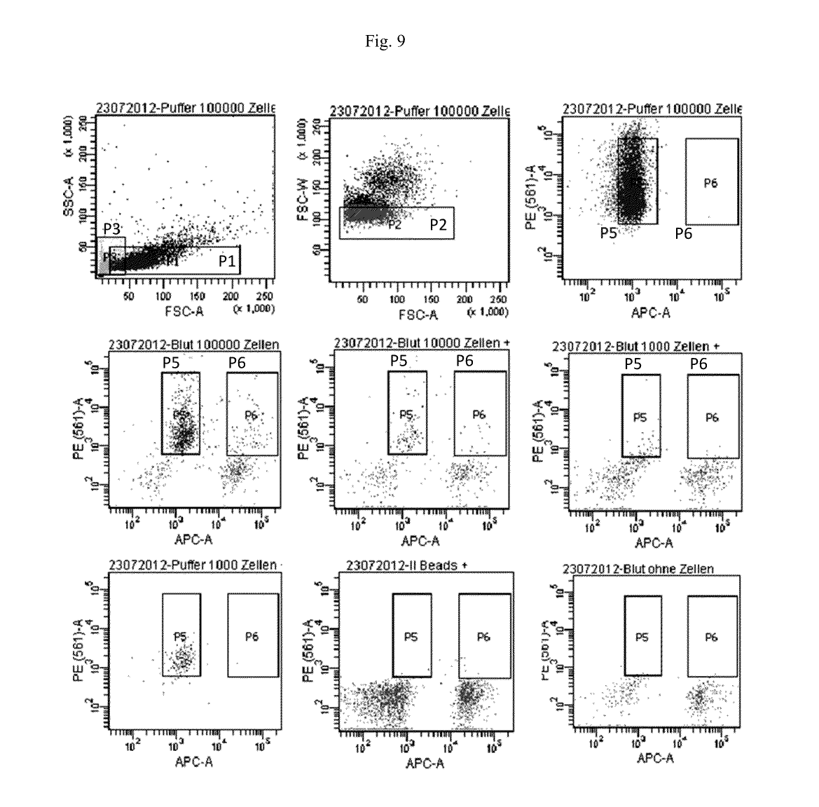

The resulting scatter diagrams shown in FIG. 9 reveal that there is a low non-specific binding of non-CTCs despite the very large excess of endogenous cells over CTCs, and that CTCs are captured efficiently from suspension as well as from whole blood: using the suggested labeling strategy, the populations are well separated in the FACS analysis. Furthermore, the fNPs are specific for CTCs as no signal above background is seen for naïve blood samples (samples without spiked cells). There is a good correlation between the number of spiked cells and the number of recovered cells, indicating that recovery is possible even at very low cell counts. Interestingly, the scatter also shows a number of EpCAM+/CD45+ (double positive) events which indicate that the labeled HCT-116 cells were also recognized by endogenous cells, presumably due to immunogenic responses.

### Example 6

### Isolation of BXPC-3 Cells from Human Whole Blood

BXPC-3 cells were prepared in PBS buffer containing 1% fetal calf serum at a concentration of 10̂6 cells/ml as described in Example 2. 10 μl of suspensions fNP-1 and fNP-2, respectively, were then added to each ml of blood.
a. Aliquots (100 μl) of BXPC-3 cell suspension containing about 100,000 cells each were added to two samples each of 1 ml citrate-stabilized human blood. Each 1.1 ml sample was incubated on ice for 30 minutes after adding 10 μl suspension of fNP-1 (See Example 3) and fNP-2 (see Example 1), respectively.
b. Aliquots (10 μl) of BXPC-3 cell suspension (10,000 cells each) were added to 1 ml aliquots of citrate-stabilized human blood. Each 1.01 ml sample was incubated on ice for 30 minutes with 10 μl of serial dilutions of fNP-1 and fNP-2, respectively.

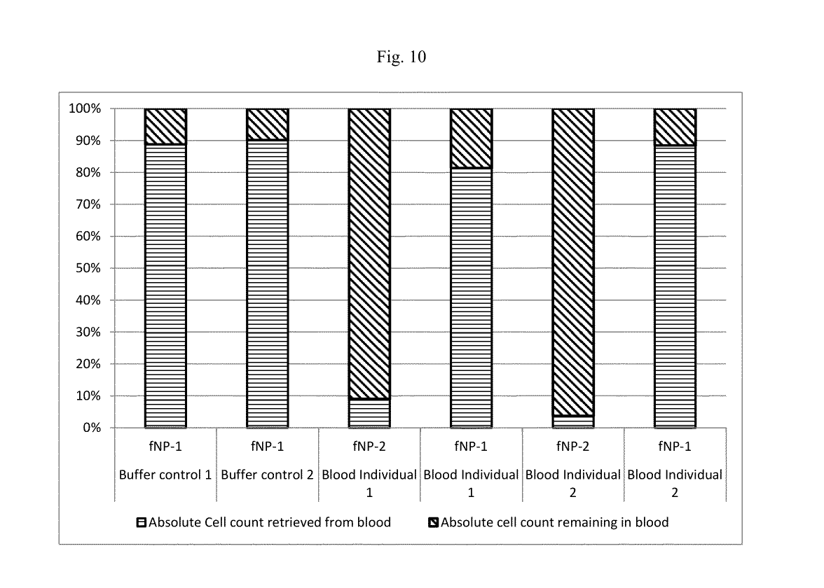

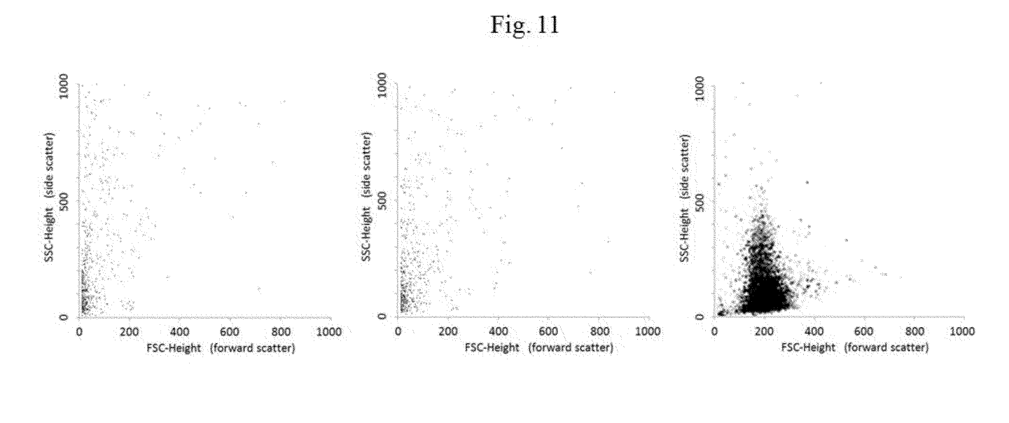

All samples from a) and b) were separately loaded onto magnetized columns filled with steel beads and pre-equilibrated with PBS/1% FCS buffer. The column was washed three times with PBS buffer and the eluate was collected. The column was removed from the magnetic field and the fNP-DACE complexes retained inside the column were eluted with 4 ml PBS buffer. 400 μl of the supernatant were then stained with 1 μl EpCAM-PE antibody (Miltenyi Order Nr. 130-096-448) (30 min on ice). 4 ml of lysing FACS buffer was added. The fixed cells were centrifuged and resuspended in 400 μl PBS for FACS analysis. 30 μl of counting beads (990 beads per μl, Bangs laboratories, Catalog Code 580, Lot. No. 10839) were added to each of the 400 μl samples. The FACS analysis was stopped once a bead count of 5,000 was reached. The resulting conversion factor for absolute quantitation of cells was 5.94. (30 μl*(990 beads/1 μl)/5,000 beads). The cell counts were corrected accordingly to yield absolute counts. The blood eluate was centrifuged (5 min. 1300 rpm Eppendorf table centrifuge) and suspended in 1.5 ml PBS/1% FCS, stained with 3 μl EpCAM-PE antibody (30 min, 0° C.), lysed for 10 min with 13.5 ml FACS lysing reagent, and centrifuged, suspended in 1 ml PBS/1% FCS and this lysing step was repeated a second time. The final cell pellet was suspended in 400 μl PBS and measured with addition of counting beads as described above. The resulting counts are depicted in FIG. 10 for samples from subset a) and in Table 3 for results from samples of subset b)

TABLE 3
Absolute cell count after spiking of about 10,000 cells into 1 ml of blood
and recovery by magnetic capture using increasing amounts (μl) of either
fNP-1 or fNP2. The absolute number of recovered cells (see column
‘Captured’) and cells found in the blood (column ‘Lost’) are listed.

| fNP-1 | Captured | Lost | % Captured |
|---|---|---|---|
| 0 μl | 216 | 7038 | 3% |
| 0.1 μl | 408 | n.a. | 6% |
| 1 μl | 6138 | 1139 | 84% |
| 5 μl | 6775 | n.a. | 99% |
| 10 μl | 6018 | 58 | 99% |
| fNP-2 | Captured | Lost | % Captured |
| 0 μl | 137 | 5119 | 3% |
| 0.1 μl | 131 | n.a. | 2% |
| 1 μl | 206 | 7209 | 3% |
| 5 μl | 1549 | 4768 | 26% |
| 10 μl | 3076 | 1517 | 67% |

Note:
Cells unaccounted for are lost in processing or due to digestion by NK-cells.

### Example 7

### Recapture of Nanoparticles from Mice after Circulation

About 100 ul of a 1% suspension of 800 nm superparamagnetic polystyrene nanoparticles (Thermo Fisher) were injected i.v. into the tail vein of three 6 month old female Balb/xid mice. After 3 hours a magnetic capture device was held to the tail of each mouse for 5 minutes. The blood flow to the tail was interrupted by finger pressure and about 2 cm of the tail tip was clipped with a new blade. The blood in the tail tip (about 0.02 ml) was removed by tail vein sampling. The beads were collected magnetically and washed 2 times with H2O and suspended in 25 ul H2O. A light scatter diagram was collected to identify the NPs (FIG. 10). Experiments were conducted with the approval of the Institutional Animal Care and Use Committee at Rider University, Lawrenceville, N.J.

### Example 8

### Evaluation of the In Vivo Distribution, Pharmacokinetics Properties, and Magnetic Capture Efficiency of Nanoparticles.

A key experiment of the process is the evaluation of the in vivo properties of nanoparticle candidates. Krukemeyer et al. (Krukemeyer, Manfred G., Veit Krenn, Martin Jakobs, and Wolfgang Wagner. “Mitoxantrone-Iron Oxide Biodistribution in Blood, Tumor, Spleen, and Liver—Magnetic Nanoparticles in Cancer Treatment.” Journal of Surgical Research 175, no. 1 (June 2012): 35-43. doi:10.1016/j.jss.2011.01.060.) and DeNardo, Sally J., Gerald L. DeNardo, Arutselvan Nataraj an, Laird A. Miers, Allan R. Foreman, Cordula Gruettner, Grete N. Adamson, and Robert Ivkov. “Thermal Dosimetry Predictive of Efficacy of 111In-ChL6 Nanoparticle AMF-Induced Thermoablative Therapy for Human Breast Cancer in Mice.” Journal of Nuclear Medicine 48, No. 3 (Mar. 1, 2007): 437-444 describes many of the experimental methods. When using nanoparticle that are functionalized at a minimum with a fluorescent label (e.g. PE FITC) as such exemplified in Example 1, it is possible to evaluate pharmacokinetic, biodistribution and capture efficiency in a single experiment, thus minimizing animal use.

In short, the procedure comprises:
a. Injection of 100-150 μl of a 1-5% suspension of magnetic beads in injection buffer into the tail vein of the 3-4 animals per group.
b. Removal of a volume of ˜50 μl-100 μl of blood from by eye bleeds or similar suitable method in after 0.5 h and 2 h after dosing.
c. Application of a laboratory magnet (a˜1 cm long magnet with an energy product of approximately 45 MGOe) for 5 min-20 min to the tail tip to collect the beads within the tail tip.
d. Removal the tail tip, removal of a second tail section (2-3 cm) and sacrifice of the animal. Recovery of about 20 μl-50 μl from the first tail element (tip) and about 100 μl from the second tail element.
e. Collection and N2 snap freeze of tissues, such as liver, spleen, and lung.

The animals are kept in their regular cages with normal access to standard diet and water and using the established light cycle during the procedure. The animals are warmed during the last 5 minutes to increase blood flow through the tail and increase the recovered tail blood volume. The blood samples are collected in a suitable collection device such as a 96-well plate preloaded with 50 μl-100 μl anticoagulant (e.g. citrate, EDTA). The plate is equipped with magnets between the wells. The magnetic force collects the nanoparticles against the wall of the wells. The remaining blood is removed by aspiration and collected separately after ˜1 min-10 min (smaller particles require the longer wait). The magnets are removed and the captured nanoparticles are suspended in 100 μl water or PBS. Suitable controls (such as a standardized amount of beads, a 100 ul aliquot of the injection formulation and blanks (injection buffer) are added as reference. A fluorescent plate reader or similar device is used to measure the amount of beads in each sample. Similarly, the about 100 mg-200 mg of each tissue is homogenized (e.g. using a bead mill) and the fluorescence intensity of the homogenates in a suitable buffer is determined. Table 4 demonstrates that as few as 1000 beads can be detected within the tissue, representing a 1:10̂7 dilution.

TABLE 4
Fluorescence signal obtained with a IVIS Spectrum Flourescence reader
from liver spiked with a series dilution of fluorescent nanoparticles
(Merck Chimie SAS, Paris, F XC 10, 100 nm XC labeled fluorescent
nanoparticles). The signal was averaged over 4 replicates. The average
background intensity is 2010. As such about 1000 particle is the lower
limit for a reliable detection in this setting.

| Dilution | Number of | Average signal - |
|---|---|---|
| factor | nanoparticles | background |
| 1 | 1E+09 | 6.87E+06 |
| 10\hat{ }-1 | 1E+08 | 8.59E+05 |
| 10\hat{ }-2 | 1E+07 | 1.58E+05 |
| 10\hat{ }-3 | 1E+06 | 4.04E+04 |
| 10\hat{ }-4 | 1E+05 | 1.50E+04 |
| 10\hat{ }-5 | 1E+04 | 7.05E+03 |
| 10\hat{ }-6 | 1E+03 | 4.41E+03 |
| 10\hat{ }-7 | 1E+02 | 3.32E+03 |
| 10\hat{ }-8 | 1E+01 | 2.75E+03 |
| 10\hat{ }-9 | 1E+00 | 2.95E+02 |
| 10\hat{ }-10 | 0E+00 | 0.00E+00 |
| 10\hat{ }-11 | 0E+00 | 0.00E+00 |

### Example 9

### Removal of DACE from the Peritoneum as a Mouse Model of Preventing Metastasis Formation in Ovarian Cancer

In a proof-of-concept study, Scarberry et al (Scarberry, Kenneth E., Erin B. Dickerson, Z. John Zhang, Benedict B. Benigno, and John F. McDonald. “Selective Removal of Ovarian Cancer Cells from Human Ascites Fluid Using Magnetic Nanoparticles.” Nanomedicine: Nanotechnology, Biology and Medicine 6, no. 3 (June 2010): 399-408. doi:10.1016/j.nano.2009.11.003.) have used a model of ovarian cancer to demonstrate that ex-vivo depletion of Ovarian cancer cells from ascites fluid significantly prolonged time to end point in a metastatic ovarian cancer model. Three groups of female C57BL/6 mice (control group I, control group II and experimental group) were intraperitoneally injected with a murine ovarian cancer cell line (ID8[VEGF160+/eGFP+]). Control group I received no intervention. MNPs were functionalized with ephrin-Al mimetic peptides selective for the EphA2 receptor that is highly expressed by several cancers. Peritoneal fluids were removed by paracentesis from the experimental group and mixed with the functionalized MNPs. Magnetic filtration was used to remove particle/malignant cell conjugates and filtered peritoneal fluids were re-introduced intraperitoneally. Control group II received the same treatment as the experimental group without MNPs. As a result, experimental group tumor progression was 10.77-times slower than that of control group I indicating that reduction of malignant cell titer significantly prolonged time to end point in a metastatic ovarian cancer model.

### Example 10

### Effect of Treatment with Functionalized Nanoparticles on Animal Survival in the 4T1-Luc2 Model of Spontaneous Metastases

The 4T1 mouse mammary tumor cell line is one of only a few breast cancer models with the capacity to metastasize efficiently to sites affected in human breast cancer. The model is well described and established and has a fast and consistent progression curve. (see: Pulaski, Beth A, Suzanne Ostrand-Rosenberg, Beth A. Pulaski, and Suzanne Ostrand-Rosenberg. “Mouse 4T1 Breast Tumor Model.” In Current Protocols in Immunology. John Wiley & Sons, Inc. Accessed Mar. 2, 2012. and Tao, Kai, Min Fang, Joseph Alroy, and G Gary Sahagian. “Imagable 4T1 Model for the Study of Late Stage Breast Cancer.” BMC Cancer 8, no. 1 (Aug. 9, 2008): 228. doi:10.1186/1471-2407-8-228.) A highly luminescent transfected 4T1 cell line, 4T1-luc2, is commercially available (Caliper LifeScience, Hopkinton, Mass.). This cell line allows the quantitative monitoring of metastasis progression.
a. Step 1: Establishment of EpCAM expression. Mouse-EpCAM-PE was obtained from Miltenyi Biotech and added to cells grown by the standard protocol. The fluorescence spectrum was recorded. The results indicated that both cell lines express murine EpCAM (Table 5).
b. Step 2: Effect of treatment with functionalized nanoparticles on animal survival in the 4T1-Luc2 model of spontaneous metastases.

The study design for a Proof-of-concept study in the 4T1 mouse model is outlined in Table 6.

TABLE 5
Cell staining with mouse EpCAM-PE

| | | Median | | |
|---|---|---|---|---|
| | | Fluorescent | Background | Percentile Expression |
| 4T1 cell | Ab | Density | subtracted | vs Parental Clone |
| Parental | no | 63.78 | | |
| 4T1-luc-2 | no | 2.07 | | |
| Parental | EpCAM | 1197.09 | 1133.31 | 100% |
| 4T1-luc-2 | EpCAM | 626.43 | 624.36 | 55% |

TABLE 6
Study design fora Proof-of-concept study in the 4T1 mouse model

| | Group 1 | Group 2 |
|---|---|---|
| Tumor Cell Line | 4T1-Luc2 (1 × 10\hat{ }4 cells injection in 50 microliter) | |
| Model | Orthotopic Model of Spontaneous Metastases | |
| Mouse Strain | BALB/c females 6-8 weeks old at the time of cell inoculation | |
| Group size | 12 mice per group | |
| Graft Site | Mammary fat pads | |
| Test Agent | Functionalized beads | Vehicle or non-functionalized |
| | | beads |

| | |
|---|---|
| Caliper measurements | Three (3) measurements prior to primary tumor resection |
| BLI of the chest area | Metastasis: once or twice weekly after primary tumor resection |
| | until the end of the study |
| Treatment Regimen | First treatment is on the day of the primary tumor resection |
| | (after the resection) and then every 3 days (a total of 3 |
| | treatments). Treatment constitutes IV injection of nanoparticles |
| | followed by a tail bleed on 3 hrs after injection. The next |
| | injection is performed within 1 hr from the bleed. A magnetic |
| | tail calf will be put on animal tails 1 hr before the tail bleed |
| Body Weight Measurements | Once before tumor inoculation. Once at the beginning of |
| and Clinical Observations | treatment. Twice per week thereafter; daily after body weight |
| | loss onset. |
| Study Duration | Pseudo-survival study (estimated 6 weeks) |

### REFERENCES

In addition, the references and GenBank accession numbers listed herein are part of the application and are incorporated by reference in their entirety as if fully set forth herein.

### U.S. Patent Documents

| | | |
|---|---|---|
| 3,970,518 | July 1976 | Giaever |
| 5,104,373 | April 1992 | Davidner et al. |
| 5,385,707 | January 1995 | Miltenyi et al. |
| 5,482,698 | January 1996 | Griffiths |
| 5,541,072 | July 1996 | Wang et al. |
| 5,646,001 | July 1997 | Terstappen et al. |
| 5,861,501 | January 1999 | Benseler et al. |
| 6,111,095 | August 2000 | Benseler et al. |
| 6,344,272 | February 2002 | Oldenburg et al. |
| 6,365,362 | April 2002 | Terstappen |
| 6,528,057 | March 2003 | Ambrus et al. |
| 6,530,944 | March 2003 | West et al. |
| 6,620,627 | September 2003 | Liberti et al. |
| 6,656,587 | December 2003 | Johnson et al. |
| 6,685,986 | February 2004 | Oldenburg et al. |
| 7,161,451 | January 2007 | Shen |
| 7,205,048 | April 2007 | Naasani |
| 7,285,412 | October 2007 | Casagrande et al. |
| 7,285,412 | October 2007 | Casagrande et al. |
| 7,998,923 | August 2011 | Pinaud et al. |
| 8,329,422 | December 2012 | Rao et al. |
| 20060141045 | June 2006 | Bhatt |
| 20060252087 | November 2006 | Tang et al. |
| 20070275007 | November 2007 | Barchi |
| 20090156976 | June 2009 | Korbling et al. |
| 20090253204 | October 2009 | King et al. |
| 20100167372 | June 2010 | King et al. |
| 20120004293 | May 2012 | Zale et al. |
| 20120230994 | September 2012 | Pan et al. |
| 2004/0191246 | September 2004 | Connelly et al. |
| 2011/0098623 | April 2011 | Zhang et al. |
| 6,417,011 | July 2002 | Miltenyi |
| 8,057,418 | November 2011 | Korbling et al. |

### Other References

1. Pantel, K., Brakenhoff, R. H. & Brandt, B. Detection, clinical relevance and specific biological properties of disseminating tumour cells. Nat. Rev. Cancer 8, 329-340 (2008).
2. GLOBOCAN: Country Fast Stat. at <http://globocan.iarc.fr/factsheets/populations/factsheet.asp?uno=900>
3. WHO|Cancer. at <http://www.who.int/mediacentre/factsheets/fs297/en/index.html>
4. Goodale, D., Phay, C., Postenka, C. O., Keeney, M. & Allan, A. L. Characterization of tumor cell dissemination patterns in preclinical models of cancer metastasis using flow cytometry and laser scanning cytometry. Cytometry A 75, 344-355 (2009).
5. Guarneri, V. & Conte, P. Metastatic breast cancer: therapeutic options according to molecular subtypes and prior adjuvant therapy. Oncologist 14, 645-656 (2009).
6. Baumann, M., Krause, M. & Hill, R. Exploring the role of cancer stem cells in radioresistance. Nat. Rev. Cancer 8, 545-554 (2008).
7. Baumann, M., Krause, M., Thames, H., Trott, K. & Zips, D. Cancer stem cells and radiotherapy. Int. J. Radiat. Biol 85, 391-402 (2009).
8. Fabian, A., Barok, M., Vereb, G. & Szollosi, J. Die hard: are cancer stem cells the Bruce Willises of tumor biology? Cytometry A 75, 67-74 (2009).
9. Shackleton, M., Quintana, E., Fearon, E. R. & Morrison, S. J. Heterogeneity in cancer: cancer stem cells versus clonal evolution. Cell 138, 822-829 (2009).
10. Pantel, K. & Brakenhoff, R. H. Dissecting the metastatic cascade. Nat. Rev. Cancer 4, 448-456 (2004).
11. Norton, L. & Massagué, J. Is cancer a disease of self-seeding? Nat. Med. 12, 875-878 (2006).
12. Kim, M.-Y. et al. Tumor self-seeding by circulating cancer cells. Cell 139, 1315-1326 (2009).
13. Norton et al 2006 Is cancer a disease of self-seeding.pdf.
14. Went, P. Frequent EpCam protein expression in human carcinomas. Human Pathology 35, 122-128 (2004).
15. Trzpis, M., McLaughlin, P. M. J., De Leij, L. M. F. H. & Harmsen, M. C. Epithelial Cell Adhesion MoleculeMore than a Carcinoma Marker and Adhesion Molecule. The American Journal of Pathology 171, 386-395 (2007).
16. Stott, S. L. et al. Isolation of circulating tumor cells using a microvortex-generating herringbone-chip. Proc Natl Acad Sci USA 107, 18392-18397 (2010).
17. Müller, V. et al. Circulating tumor cells in breast cancer: correlation to bone marrow micrometastases, heterogeneous response to systemic therapy and low proliferative activity. Clin. Cancer Res 11, 3678-3685 (2005).
18. Cristofanilli, M. et al. Circulating tumor cells in metastatic breast cancer: biologic staging beyond tumor burden. Clin. Breast Cancer 7, 471-479 (2007).
19. Budd, G. T. et al. Circulating tumor cells versus imaging—predicting overall survival in metastatic breast cancer. Clin. Cancer Res 12, 6403-6409 (2006).
20. Cristofanilli, M. et al. Circulating tumor cells: a novel prognostic factor for newly diagnosed metastatic breast cancer. J. Clin. Oncol 23, 1420-1430 (2005).
21. Braun, S. et al. Cytokeratin-positive cells in the bone marrow and survival of patients with stage I, II, or III breast cancer. N. Engl. J. Med 342, 525-533 (2000).
22. Yagata, H. et al. Evaluation of circulating tumor cells in patients with breast cancer: multi-institutional clinical trial in Japan. Int. J. Clin. Oncol 13, 252-256 (2008).
23. Dawood, S. & Cristofanilli, M. Integrating circulating tumor cell assays into the management of breast cancer. Curr Treat Options Oncol 8, 89-95 (2007).
24. Tan, L. K. et al. Occult Axillary Node Metastases in Breast Cancer Are Prognostically Significant: Results in 368 Node-Negative Patients With 20-Year Follow-Up. Journal of Clinical Oncology 26, 1803-1809 (2008).
25. Bidard, F.-C. et al. Prognosis of women with stage 1V breast cancer depends on detection of circulating tumor cells rather than disseminated tumor cells. Ann. Oncol 19, 496-500 (2008).
26. Hayes, D. F. et al. Circulating tumor cells at each follow-up time point during therapy of metastatic breast cancer patients predict progression-free and overall survival. Clin. Cancer Res 12, 4218-4224 (2006).
27. Cancer Facts & Figures. 2011. at <http://www.cancer.org/Research/CancerFactsFigures/CancerFactsFigures/cancer-facts-figures-2011>
28. Shubayev, V. I., Pisanic, T. R., 2nd & Jin, S. Magnetic nanoparticles for theragnostics. Adv. Drug Deliv. Rev. 61, 467-477 (2009).
29. Pankhurst, Q. A., Connolly, J., Jones, S. K. & Dobson, J. Applications of magnetic nanoparticles in biomedicine. Journal of Physics D: Applied Physics 36, R167-R181 (2003).
30. Tassa, C., Shaw, S. Y. & Weissleder, R. Dextran-Coated Iron Oxide Nanoparticles: a Versatile Platform for Targeted Molecular Imaging, Molecular Diagnostics and Therapy. Acc Chem Res 44, 842-852 (2011).
31. Ambrogio, M. W., Thomas, C. R., Zhao, Y.-L., Zink, J. I. & Stoddart, J. F. Mechanized Silica Nanoparticles: A New Frontier in Theranostic Nanomedicine. Acc. Chem. Res. 44, 903-913 (2011).
32. Beaudette, T. T. et al. Chemoselective Ligation in the Functionalization of Polysaccharide-based Particles. J Am Chem Soc 131, 10360-10361 (2009).
33. Byrne, J. D., Betancourt, T. & Brannon-Peppas, L. Active targeting schemes for nanoparticle systems in cancer therapeutics. Advanced Drug Delivery Reviews 60, 1615-1626 (2008).
34. Castaneda, R. T., Khurana, A., Khan, R. & Daldrup-Link, H. E. Labeling Stem Cells with Ferumoxytol, an FDA-Approved Iron Oxide Nanoparticle. J Vis Exp (2011).doi:10.3791/3482
35. Herrmann, I. K., Grass, R. N. & Stark, W. J. High-strength metal nanomagnets for diagnostics and medicine: carbon shells allow long-term stability and reliable linker chemistry. Nanomedicine (Lond) 4, 787-798 (2009).
36. Ho, D., Sun, X. & Sun, S. Monodisperse Magnetic Nanoparticles for Theranostic Applications. Acc. Chem. Res. 44, 875-882 (2011).
37. Leonard, F. Imaging and cancer: A review. Molecular Oncology 2, 115-152 (2008).
38. Jokerst, J. V., Lobovkina, T., Zare, R. N. & Gambhir, S. S, Nanoparticle PEGylation for imaging and therapy. Nanomedicine (Lond) 6, 715-728 (2011).
39. Lin, M. M., Kim, D. K., El Haj, A. J. & Dobson, J. Development of superparamagnetic iron oxide nanoparticles (SPIONS) for translation to clinical applications. IEEE Trans Nanobioscience 7, 298-305 (2008).
40. Low, K., Wacker, M., Wagner, S., Langer, K. & Von Briesen, H. Targeted human serum albumin nanoparticles for specific uptake in EGFR-Expressing colon carcinoma cells. Nanomedicine: Nanotechnology, Biology and Medicine 7, 454-463 (2011).
41. Ng, K. K., Lovell, J. F. & Zheng, G. Lipoprotein-Inspired Nanoparticles for Cancer Theranostics. Acc. Chem. Res. 44, 1105-1113 (2011).
42. Nie, Y., Zhang, Z.-R., He, B. & Gu, Z. Investigation of PEG-PLGA-PEG Nanoparticles-based Multipolyplexes for IL-18 Gene Delivery. J Biomater Appl (2011).doi:10.1177/0885328210384889
43. Suh, J. et al. PEGylation of nanoparticles improves their cytoplasmic transport. Int J Nanomedicine 2, 735-741 (2007).
44. Sussman, E. M., Clarke, M. B., Jr & Shastri, V. P. Single-step process to produce surface-functionalized polymeric nanoparticles. Langmuir 23, 12275-12279 (2007).
45. Te Velde, E. A., Veerman, T., Subramaniam, V. & Ruers, T. The use of fluorescent dyes and probes in surgical oncology. European Journal of Surgical Oncology (EJSO) 36, 6-15 (2010).
46. Veiseh, 0., Gunn, J. W. & Zhang, M. Design and fabrication of magnetic nanoparticles for targeted drug delivery and imaging. Adv. Drug Deliv. Rev. 62, 284-304 (2010).
47. Vigor, K. L. et al. Nanoparticles functionalized with recombinant single chain Fv antibody fragments (scFv) for the magnetic resonance imaging of cancer cells. Biomaterials 31, 1307-1315 (2010).
48. Vij, N. et al. Development of PEGylated PLGA nanoparticle for controlled and sustained drug delivery in cystic fibrosis. Journal of Nanobiotechnology 8, 22 (2010).
49. Wang, Y., Dave, R. N. & Pfeffer, R. Polymer coating/encapsulation of nanoparticles using a supercritical anti-solvent process. The Journal of Supercritical Fluids 28, 85-99 (2004).
50. Yoo, M. K. et al. Superparamagnetic iron oxide nanoparticles coated with mannan for macrophage targeting. J Nanosci Nanotechnol 8, 5196-5202 (2008).
51. DeNardo, S. J. et al. Thermal Dosimetry Predictive of Efficacy of 111In-ChL6 Nanoparticle AMF-Induced Thermoablative Therapy for Human Breast Cancer in Mice. J Nucl Med 48, 437-444 (2007).
52. Ernsting, M. J., Tang, W.-L., MacCallum, N. W. & Li, S.-D. Preclinical pharmacokinetic, biodistribution, and anti-cancer efficacy studies of a docetaxel-carboxymethylcellulose nanoparticle in mouse models. Biomaterials 33, 1445-1454 (2012).
53. Gutierrez, L. et al. Magnetic Nanoparticle Location and Quantification in Mice Tissues after Intravenous Injection. AIP Conference Proceedings 1275, 141-144 (2010).
54. Khemtong, C. et al. In vivo Off-Resonance Saturation Magnetic Resonance Imaging of αvβ3-Targeted Superparamagnetic Nanoparticles. Cancer Res 69, 1651-1658 (2009).
55. Richter, H. et al. Magnetorelaxometric quantification of magnetic nanoparticles in an artery model after ex vivo magnetic drug targeting. Phys Med Biol 54, N417-424 (2009).
56. Scarberry, K. E., Dickerson, E. B., McDonald, J. F. & Zhang, Z. J. Magnetic Nanoparticle—Peptide Conjugates for in Vitro and in Vivo Targeting and Extraction of Cancer Cells. Journal of the American Chemical Society 130, 10258-10262 (2008).
57. Scarberry, K. E., Mezencev, R. & McDonald, J. F. Targeted removal of migratory tumor cells by functionalized magnetic nanoparticles impedes metastasis and tumor progression. Nanomedicine (Lond) 6, 69-78 (2011).
58. Scarberry, K. E., Dickerson, E. B., Zhang, Z. J., Benigno, B. B. & McDonald, J. F. Selective removal of ovarian cancer cells from human ascites fluid using magnetic nanoparticles. Nanomedicine: Nanotechnology, Biology and Medicine 6, 399-408 (2010).
59. Herrmann, I. K. et al. Device for continuous extracorporeal blood purification using target-specific metal nanomagnets. Nephrol. Dial. Transplant. 26, 2948-2954 (2011).
60. Herrmann, I. K. et al. Blood Purification Using Functionalized Core/Shell Nanomagnets. Small 6, 1388-1392 (2010).
61. John, R. & Boppart, S. A. Magnetomotive molecular nanoprobes. Curr. Med. Chem. 18, 2103-2114 (2011).
62. Kruttwig, K. et al. Development of a three-dimensional in vitro model for longitudinal observation of cell behavior: monitoring by magnetic resonance imaging and optical imaging. Mol Imaging Biol 12, 367-376 (2010).
63. Slotkin, J. R., Cahill, K. S., Tharin, S. A. & Shapiro, E. M. Cellular magnetic resonance imaging: nanometer and micrometer size particles for noninvasive cell localization. Neurotherapeutics 4, 428-433 (2007).
64. Galanzha, E. I. et al. In vivo magnetic enrichment and multiplex photoacoustic detection of circulating tumour cells. Nat Nano 4, 855-860 (2009).
65. Fadeel, B. & Garcia-Bennett, A. E. Better safe than sorry: Understanding the toxicological properties of inorganic nanoparticles manufactured for biomedical applications. Advanced Drug Delivery Reviews 62, 362-374 (2010).
66. Metsälä, M., Gilijamse, J. J., Hoekstra, S., Van de Meerakker, S. Y. T. & Meijer, G. Reflection of OH molecules from magnetic mirrors. New Journal of Physics 10, 053018 (2008).
67. Robertson, W., Cazzolato, B. & Zander, A. Parameters for Optimizing the Forces Between Linear Multipole Magnet Arrays. IEEE Magnetics Letters 1, 0500304 (2010).
68. Zanini, L. F., Dempsey, N. M., Givord, D., Reyne, G. & Dumas-Bouchiat, F. Autonomous micro-magnet based systems for highly efficient magnetic separation. Applied Physics Letters 99, 232504-232504-3 (2011).
69. Grüttner, C. et al. Synthesis and antibody conjugation of magnetic nanoparticles with improved specific power absorption rates for alternating magnetic field cancer therapy. Journal of Magnetism and Magnetic Materials 311, 181-186 (2007).
70. Topalian, S. L. et al. Safety, Activity, and Immune Correlates of Anti-PD-1 Antibody in Cancer. New England Journal of Medicine 366, 2443-2454 (2012).

1. A method for removal of disease-associated circulating entities from an organism, comprising:
a. introducing paramagnetic or superparamagnetic nanoparticles into a fluid component of the organism, wherein the nanoparticles are functionalized so that they comprise at least one bioaffinity molecule that binds to a first target surface structure on a first disease-associated circulating entity and wherein the nanoparticles are introduced into a compartment of the body that permits the nanoparticles to contact the first disease-associated circulating entities, wherein complexes of functionalized nanoparticles and first disease-associated circulating entities form upon contact;
b. applying a magnetic field to a target region of the body, wherein the magnetic field concentrates the nanoparticles within the target region; and
c. removing from the body the complexes within the target region.

2. The method of claim 1 wherein the magnetic field of step b is externally applied.

3. The method of claim 1 wherein the contacting of the first disease-associated circulating entities and the nanoparticles is enhanced by an additional step of magnetic mixing of the first disease-associated circulating entities and the nanoparticles.

4. The method of claim 3 wherein the magnetic mixing step comprises an externally applied magnetic field wherein the magnetic field causes relative motion of the nanoparticles with respect to the fluid within which the nanoparticles circulate and wherein the intensity of the magnetic field does not immobilize the nanoparticles.

5. The method of claim 1 wherein the step of applying a magnetic field is accomplished using a magnetic capture device that applies a magnetic field of sufficient intensity within the target region to immobilize the complexes in the fluid.

6. The method of claim 5 wherein the step of removing the complexes is accomplished by removing the fluid within the target region with a removal device.

7. The method of claim 6 wherein the removal device is a syringe or a cannula.

8. The method of claim 6 wherein the fluid is blood and wherein the flow of blood into the target region is temporarily halted.

9. The method of claim 1 wherein the disease-associated circulating entities are a first population of cells of the organism, wherein the first target surface structure on the first population of cells distinguishes the first population of cells from other populations of cells in the organism.

10. The method of claim 1 wherein the first disease-associated circulating entity comprises a plurality of different target surface structures and the nanoparticles are functionalized so as to comprise a plurality of different bioaffinity molecules, wherein each different bioaffinity molecule binds specifically to one of the plurality of different target surface structures on the first disease-associated circulating entity.
